(19) United States
(12) Patent Application Publication
(10) Pub. No.: US 2023/0085214 A1
(43) Pub. Date: Mar. 16, 2023

(54) METHODS FOR LABELING AND TARGETING CELLS

(71) Applicants: President and Fellows of Harvard College, Cambridge, MA (US); Whitehead Institute for Biomedical Research, Cambridge, MA (US)

(72) Inventors: Hua Wang, Cambridge, MA (US); Andrew Salim Khalil, Cambridge, MA (US); David J. Mooney, Sudbury, MA (US); Rudolf Jaenisch, Cambridge, MA (US)

(73) Assignees: President and Fellows of Harvard College, Cambridge, MA (US); Whitehead Institute for Biomedical Research, Cambridge, MA (US)

(21) Appl. No.: 17/869,611

(22) Filed: Jul. 20, 2022

Related U.S. Application Data
(63) Continuation of application No. PCT/US2021/015912, filed on Jan. 29, 2021.
(60) Provisional application No. 62/967,387, filed on Jan. 29, 2020.

(51) Int. Cl. A61K 47/54; C12N 15/11; C12N 9/22; C12N 15/90

(52) U.S. Cl. CPC A61K 47/555; C12N 15/11; C12N 9/22; C12N 15/907; A61K 47/549; C12N 2310/20; C12N 2800/80; C12N 2310/3517; C12N 2320/32; C07K 2319/60

(57) ABSTRACT

Disclosed herein are compositions and methods for labeling cells using click chemistry reagents. The compositions and methods disclosed herein provide a specific and efficient means of localizing desired agents to a variety of cell types in vivo and in vitro. The compositions and methods disclosed herein can be used to deliver a variety of desired agents to a cell or population of cells to direct cell fate and/or cell differentiation.

### RELATED APPLICATIONS

This application is a continuation of International Patent Application No. PCT/US2021/015912, filed on Jan. 29, 2021, which claims priority to and benefit of U.S. Provisional Patent Application No. 62/967,387, filed on Jan. 29, 2020. The entire contents of each of these applications are incorporated herein by reference in their entirety.

### GOVERNMENT SUPPORT

This invention was made with Government support under EB023287, CA214369, and CA223255 awarded by the National Institutes of Health. The Government has certain rights in the invention.

### BACKGROUND OF THE INVENTION

There are many challenges inherent in labeling and/or targeting specific cell types. Antibodies are commonly used for cell-specific targeting, however, this strategy requires identification of cell-specific antigens, and accessibility of antibodies to the target cells, and can be limited by internalization of the antibody and its cognate antigen from the cell surface. Accordingly, there is a need for method to label and target cells.

### SUMMARY OF THE INVENTION

Disclosed herein are methods for metabolically labeling and targeting cells using click chemistry reagents. The methods disclosed herein provide a specific and efficient means of delivering desired agents to a variety of cell types (FIGS. 1A-1D). In some embodiments, delivery of a desired agent to a cell can be used to label a cell. In some embodiments, delivery of a desired agent to a cell can be used to direct cell fate and/or cell differentiation. For example, cell fate and/or cell differentiation may be directed through precise control, sequential, and/or repetitive delivery to a cell of a variety of agents, such as polypeptides, signaling molecules, and/or nucleic acids. In some embodiments, delivery of a desired agent to a cell to direct cell fate and/or cell differentiation can comprise the preceise control of intercellular levels of the desired agent. In certain embodiments, different, orthogonal click-functional groups can be utilized to deliver different agents to a variety of cell types for controlling cell fates of mixed cell populations, e.g., simultaneously or sequentially. In certain embodiments, controlled differentiation of a heterogenous stem cell pool and/or a single-type cell pool may be achieved via orthogonal targeting of different active agents to a cell or a plurality of cells.

Accordingly, in one aspect, the present invention provides a method of delivering an agent to a cell intracellularly. The method includes contacting the cell coupled to a first click reagent with the agent coupled to a second click reagent, wherein the second click reagent selectively reacts with the first click reagent coupled to the cell, thereby delivering the agent to the cell intracellularly. In one embodiment, the agent retains its structural integrity, function and/or activity after delivery to the cell intracellularly. In some embodiments, the cell is coupled to a plurality of click reagents, optionally, wherein the plurality of click reagents are of the same type and/or are of different types. In some embodiments, the cell is contacted with the agent coupled to a second click reagent a plurality of times.

In some embodiments, the method includes contacting the cell with an additional agent coupled to a click reagent. In some embodiments, the additional agent coupled to a click reagent binds to the same click reagent coupled to the cell as the agent. In some embodiments, the additional agent coupled to a click reagent does not bind to the same click reagent coupled to the cell as the agent. Instead, in some embodiments, the additional agent coupled to a click reagent can bind to a click reagent coupled to the cell that is different from that which the agent binds.

For example, in some embodiments, the method includes contacting the cell with an additional agent coupled to the second click reagent. In some embodiments, the additional agent coupled with the second click reagent contacts the cell prior to, concurrently with, or subsequent to contacting the cell with the agent coupled with the second click reagent. In one embodiment, the additional agent coupled with the second click reagent retains its structural integrity, function and/or activity after delivery to the cell intracellularly. In some embodiments, the cell is contacted with the additional agent coupled with the second click reagent a plurality of times.

In some embodiments, at least one second agent coupled to a click reagent selectively reacts with a click reagent on a second cell. For example, in some embodiments, a second agent coupled to a third click reagent selectively reacts with a fourth click reagent on a second cell. In one embodiment, the second agent coupled with the third click reagent retains its structural integrity, function and/or activity after delivery to the cell intracellularly. In some embodiments, the cell is contacted with the second agent coupled with the third click reagent a plurality of times. Accordingly, the methods disclosed herein provide a specific and efficient means of delivering multiple agents (e.g., at least two agents, at least three agents, at least four agents, at least 5 agents, or more) to a variety of cells, optionally, wherein the cells are of the same type or are of different types.

In some embodiments, the cell is further coupled to a third click reagent, and the method further comprises contacting the cell with an agent coupled to a fourth click reagent, wherein the fourth click reagent selectively reacts with the third click reagent. In some embodiments, the agent coupled to the fourth click reagent contacts the cell prior to, concurrently with, or subsequent to contacting the cell with the agent coupled with the second click reagent. In one embodiment, the agent coupled with the fourth click reagent retains its structural integrity, function and/or activity after delivery to the cell intracellularly. In some embodiments, the cell is contacted with the agent coupled with the fourth click reagent a plurality of times.

In some embodiments, the method includes contacting the cell with an additional agent coupled to the fourth click reagent. In some embodiments, the additional agent coupled with the fourth click reagent contacts the cell prior to, concurrently with, or subsequent to contacting the cell with the agent coupled with the second click reagent and/or the additional agent coupled with the second click reagent. In one embodiment, the additional agent coupled with the fourth click reagent retains its structural integrity, function and/or activity after delivery to the cell intracellularly. In some embodiments, the cell is contacted with the additional agent coupled with the fourth click reagent a plurality of times.

In some embodiments, at least one second agent coupled to a fifth click reagent selectively reacts with a sixth click reagent on a second cell.

In some embodiments, the cell is further coupled to a fifth click reagent, and the method further comprises contacting the cell with an agent coupled to a sixth click reagent, wherein the sixth click reagent selectively reacts with the fifth click reagent. In some embodiments, the agent coupled to the sixth click reagent contacts the cell prior to, concurrently with, or subsequent to contacting the cell with the agent coupled with the fourth click reagent. In one embodiment, the agent coupled with the sixth click reagent retains its structural integrity, function and/or activity after delivery to the cell intracellularly. In some embodiments, the cell is contacted with the agent coupled with the sixth click reagent a plurality of times.

In one embodiment, the method includes contacting the cell with an additional agent coupled to the sixth click reagent. In one embodiments, the additional agent coupled with the sixth click reagent contacts the cell prior to, concurrently with, or subsequent to contacting the cell with the agent coupled with the second click reagent, the additional agent coupled with the second click reagent, the agent coupled with the fourth click reagent, and/or the additional agent coupled with the fourth click reagent. In one embodiment, the additional agent coupled with the sixth click reagent retains its structural integrity, function and/or activity after delivery to the cell intracellularly. In one embodiment, the cell is contacted with the additional agent coupled with the sixth click reagent a plurality of times.

In some embodiments, at least one second agent coupled to a seventh click reagent selectively reacts with an eighth click reagent on a second cell.

In some embodiments, the cell is in a plurality of cells (e.g., a population of cells). For example, the cell may be in a heterogenous stell cell pool, or the cell may be in a single-type stem cell pool.

In another aspect, the present invention provides a method of selectively delivering an agent to a cell in a plurality of cells intracellularly, wherein the cell is coupled with a first click reagent, comprising contacting the plurality of cells with the agent coupled to a second click reagent, wherein the second click reagent selectively reacts with the first click reagent coupled to the cell, thereby delivering the agent selectively to the cell intracellularly. In one embodiment, the agent coupled with the second click reagent retains its structural integrity, function and/or activity after delivery to the cell intracellularly. In some embodiments, the plurality of cells is contacted with the agent coupled with the second click reagent a plurality of times.

In some embodiments, the method includes contacting the plurality of cells with an additional agent coupled to the second click reagent. In some embodiments, the additional agent coupled with the second click reagent contacts the plurality of cells prior to, concurrently with, or subsequent to contacting the plurality of cells with the agent coupled with the second click reagent. In one embodiment, the additional agent coupled with the second click reagent retains its structural integrity, function and/or activity after delivery to the cell intracellularly. In some embodiments, the plurality of cells is contacted with the additional agent coupled with the second click reagent a plurality of times.

In some embodiments, the cell is further coupled to a third click reagent, and the method further comprises contacting the plurality of cells with an agent coupled to a fourth click reagent, wherein the fourth click reagent selectively reacts with the third click reagent. In some embodiments, the agent coupled to the fourth click reagent contacts the plurality of cells prior to, concurrently with, or subsequent to contacting the plurality of cells with the agent coupled with the second click reagent. In one embodiment, the agent coupled with the fourth click reagent retains its structural integrity, function and/or activity after delivery to the cell intracellularly. In some embodiments, the plurality of cells is contacted with the agent coupled with the fourth click reagent a plurality of times.

In some embodiments, the method includes contacting the plurality of cells with an additional agent coupled to the fourth click reagent. In some embodiments, the additional agent coupled with the fourth click reagent contacts the plurality of cells prior to, concurrently with, or subsequent to contacting the plurality of cells with the agent coupled with the second click reagent and/or the additional agent coupled with the second click reagent. In one embodiment, the additional agent coupled with the fourth click reagent retains its structural integrity, function and/or activity after delivery to the cell intracellularly. In some embodiments, the plurality of cells is contacted with the additional agent coupled with the fourth click reagent a plurality of times.

In some embodiments, the cell is further coupled to a fifth click reagent, and the method further comprises contacting the plurality of cells with an agent coupled to a sixth click reagent, wherein the sixth click reagent selectively reacts with the fifth click reagent. In some embodiments, the agent coupled to the sixth click reagent contacts the plurality of cells prior to, concurrently with, or subsequent to contacting the plurality of cells with the agent coupled with the fourth click reagent. In one embodiment, the agent coupled with the sixth click reagent retains its structural integrity, function and/or activity after delivery to the cell intracellularly. In some embodiments, the plurality of cells is contacted with the agent coupled with the sixth click reagent a plurality of times.

In one embodiment, the method includes contacting the plurality of cells with an additional agent coupled to the sixth click reagent. In one embodiment, the additional agent coupled with the sixth click reagent contacts the plurality of cells prior to, concurrently with, or subsequent to contacting the plurality of cells with the agent coupled with the second click reagent, the additional agent coupled with the second click reagent, the agent coupled with the fourth click reagent, and/or the additional agent coupled with the fourth click reagent. In one embodiment, the additional agent coupled with the sixth click reagent retains its structural integrity, function and/or activity after delivery to the cell intracellularly. In one embodiment, the plurality of cells is contacted with the additional agent coupled with the sixth click reagent a plurality of times.

In some embodiments, the plurality of the cells (e.g., population of cells) are homogenous.

In some embodiments, the plurality of the cells (e.g., population of cells) are heterogenous.

In some embodiments, the plurality of cells (e.g., population of cells) are coupled to a plurality of click reagents. In some embodiments, the agent and/or the additional agent induces the differentiation of the cell and/or the plurality of cells (e.g., population of cells) coupled with a plurality of click reagents.

In some embodiments, the agent and/or the additional agent induces the differentiation of the cell coupled with a click reagent and/or the plurality of cells (e.g., population of cells) coupled with a click reagent. In some embodiments, the agent and/or the additional agent induces the differentiation of the cell coupled with the first click reagent. In some embodiments, the agent and/or the additional agent induces the differentiation of the cell coupled with the third click reagent. In some embodiments, the agent and/or the additional agent induces the differentiation of the cell coupled with the fifth click reagent.

In some embodiments, the agent and/or the additional is a cell differentiation inducing agent.

In another embodiment, the click reagent is selected from the group consisting of azide, dibenzocyclooctyne (DBCO), transcyclooctene, tetrazine, norbornene, and variants thereof. In another embodiment, the click reagent/the second click agent pair is selected from the group consisting of azide/dibenzocyclooctyne (DBCO), tetrazine/trans-cyclooctene, tetrazine/norbornene, trans-cyclooctene/tetrazine, norbornene/tetrazine, and variants thereof. In still another embodiment, the first click reagent is azide and the second click reagent is dibenzocyclooctyne (DBCO).

In yet another embodiment, the agent and/or the additional agent is independently selected from the group consisting of a small molecule, a nucleic acid, a protein or a peptide, and any combination thereof.

In one embodiment, the agent and/or the additional agent comprises a protein or a peptide. In another embodiment, the protein or peptide is selected from the group consisting of a transcriptional factor, a growth factor, a cytokine, an antibody, and a gene editing protein or peptide. In still another embodiment, the protein or peptide is a transcriptional factor that modulates the expression of one or more genes. In yet another embodiment, the protein or peptide is a gene editing protein or peptide that edits or modifies a gene or the genome of the cell. In another embodiment, the gene editing protein or peptide is selected from the group consisting of meganuclease, zinc finger nuclease (ZFN), transcription ctivator-like effector-based nuclease (TALEN), and CRISPR associate protein (Cas). In still another embodiment, the protein or peptide is a CRISPR associate protein 9 (Cas9).

In one embodiment, the agent and/or the additional agent comprises a nucleic acid. In another embodiment, the nucleic acid encodes a protein or a peptide. In still another embodiment, the nucleic acid modulates the expression of a gene. In yet another embodiment, the nucleic acid is selected from the group consisting of siRNA, shRNA, ribozyme RNA, iRNA, sgRNA, and miRNA.

In one embodiment, the cell and/or the plurality of cells is selected from the group consisting of a stem cell, a primary cell, a neural cell, and a fibroblast cell.

In some embodiments, the cell and/or the plurality of cells (e.g., population of cells) are stem cells, optionally, selected from the group consisting of embryonic stem cells (ESCs), induced pluripotent stem cells, mobilized peripheral blood stem cells, astrocyte, blastocoel, blastocyst, bone marrow stromal cells, cord blood stem cells, hematopoietic stem cells, mesenchymal stem cells, neural stem cells, somatic stem cells, and trophoblast stem cells.

In some embodiments, the cell and/or the plurality of cells (e.g., population of cells) are primary cells.

In one aspect, the present disclosure provides a method of delivering an agent to a cell intracellularly. The method includes contacting the cell coupled to an azide with the agent coupled to dibenzocyclooctyne (DBCO), thereby delivering the agent to the cell intracellularly, wherein the agent retains its structural integrity, function and/or activity while residing within the cell.

In another aspect, the present invention provides a method of delivering a CRISPR associated protein 9 (Cas9) to a cell intracellularly. The method includes contacting the cell coupled to an azide with the Cas9 coupled to dibenzocyclooctyne (DBCO), thereby delivering the Cas9 to the cell intracellularly, wherein the Cas9 retains its structural integrity, function and/or activity while residing within the cell.

In one embodiment, the agent is delivered to the cell in vitro. In another embodiment, the agent is delivered to the cell in vivo in a subject, comprising administering to the subject the cell coupled or to be coupled to the first click agent and the agent coupled to the second click agent. In another embodiment, the agent is administered to the subject prior to, concurrently with, or after the administration of the cell coupled to the click agent. In still another embodiment, the cell is coupled in vitro and administered to the subject in the absence of a scaffold. In yet another embodiment, the cell is coupled in vivo within a scaffold.

In one aspect, the present invention provides a method of editing or modifying a gene or the genome of a cell. The method includes contacting the cell which is coupled to a first click reagent with a gene editing molecule coupled to a second click reagent; wherein the second click reagent selectively reacts with the first click reagent coupled to the cell, thereby editing or modifying the gene or the genome of the cell. In one embodiment, the first click reagent is selected from the group consisting of azide, dibenzocyclooctyne (DBCO), transcyclooctene, tetrazine, norbornene, and variants thereof. In still another embodiment, the first click reagent is azide and the second click reagent is dibenzocyclooctyne (DBCO).

In one embodiment, the gene editing molecule is selected from the group consisting of meganuclease, zinc finger nuclease (ZFN), transcription ctivator-like effector-based nuclease (TALEN), and CRISPR associate protein (Cas). In another embodiment, the gene editing molecule is CRISPR associate protein 9 (Cas9).

In still another embodiment, the gene editing molecule is a nucleic acid encoding a protein or peptide. In yet another embodiment, the protein or peptide is selected from the group consisting of meganuclease, zinc finger nuclease (ZFN), transcription activator-like effector-based nuclease (TALEN), and CRISPR associate protein (Cas). In another embodiment, the protein or peptide is CRISPR associate protein 9 (Cas9).

In one embodiment, the method further includes contacting the cell with a single guide RNA (sgRNA).

In one embodiment, the gene editing molecule contacts the cell in vitro. In another embodiment, the gene editing molecule is targeted to the cell in vivo in a subject, comprising administering the subject the cell coupled or to be coupled to the first click agent and the gene editing molecule coupled to the second click agent. In still another embodiment, the gene editing molecule is administered to the subject prior to, concurrently with, or after the administration of the cell coupled to the first click agent.

In another embodiment, the cell is coupled in vitro and administered to the subject in the absence of a scaffold. In still another embodiment, the cell is coupled in vivo within a scaffold.

In one aspect, the present invention provides a method of inducing differentiation of a cell and/or a plurality of cells (e.g., a population of cells) in vivo or in vitro. The method includes contacting the cell and/or the plurality of cells (e.g., a population of cells) which is coupled with a first click reagent with a cell differentiation inducing agent coupled to a second click reagent; wherein the second click reagent selectively reacts with the first click reagent coupled to the cell, thereby inducing the differentiation of the cell. In one embodiment, the cell differentiation inducing agent is present within the cell, and wherein the cell differentiation inducing agent retains its structural integrity, function and/or activity while residing within the cell. In some embodiments, the method includes contacting the cell, or the plurality of cells, which is coupled with one or more click reagents (e.g., 1, 2, 3, 4, 5, 6, 7, 8, 9, 10, or more click reagents) with one or more cell differentiation inducing agents (e.g., 1, 2, 3, 4, 5, 6, 7, 8, 9, 10, or more cell differentiation inducing agents) coupled to one or more click reagents (e.g., 1, 2, 3, 4, 5, 6, 7, 8, 9, 10, or more click reagents).

In another embodiment, the first click reagent is selected from the group consisting of azide, dibenzocyclooctyne (DBCO), transcyclooctene, tetrazine, norbornene, and variants thereof. In still another embodiment, the first click reagent is azide and the second click reagent is dibenzocyclooctyne (DBCO).

In one embodiment, the cell differentiation inducing agent is a transcriptional regulator or a gene editing molecule. In another embodiment, the cell differentiation inducing agent is a gene editing molecule selected from the group consisting of meganuclease, zinc finger nuclease (ZFN), transcription ctivator-like effector-based nuclease (TALEN), and CRISPR associate protein (Cas). In still another embodiment, the method further includes contacting the cell with a single guide RNA (sgRNA).

In another embodiment, the cell is selected from the group consisting of a stem cell, a primary cell, a neural cell, and a fibroblast cell.

In still another embodiment, the differentiation inducing agent contacts the cell in vitro. In yet another embodiment, the differentiation inducing agent is targeted to the cell in vivo in a subject, comprising administering the subject cell coupled or to be coupled to the first click agent and the differentiation inducing agent coupled to the second click agent.

In one embodiment, the differentiation inducing agent is administered to the subject prior to, concurrently with, or after the administration of the cell coupled or to be coupled to the first click agent.

In another embodiment, the cell is coupled in vitro and administered to the subject in the absence of a scaffold. In still another embodiment, the cell is coupled in vivo within a scaffold.

In one aspect, the present disclosure provides a cell. The cell includes a glycoprotein coupled to a first click agent; and an agent coupled to a second click agent, wherein the glycoprotein is covalently linked to the agent through a selective reaction between the first click agent and the second click agent, wherein the glycoprotein-agent complex is located within the cell, and wherein the agent retains its structural integrity, function or activity.

In one embodiment, the first click agent is selected from the group consisting of azide, dibenzocyclooctyne (DBCO), transcyclooctene, tetrazine, norbornene, and variants thereof. In another embodiment, the click reagent/the second click agent pair is selected from the group consisting of azide/dibenzocyclooctyne (DBCO), tetrazine/trans-cyclooctene, tetrazine/norbornene, trans-cyclooctene/tetrazine, norbornene/tetrazine, and variants thereof.

In another embodiment, the agent is selected from the group consisting of a small molecule, a nucleic acid, a protein or a peptide, and any combination thereof.

In still another embodiment, the agent comprises a protein or a peptide. In yet another embodiment, the protein or peptide is selected from the group consisting of a transcriptional factor, a growth factor, a cytokine, an antibody, and a gene editing protein or peptide. In another embodiment, the protein or peptide is a transcriptional factor that modulates the expression of one or more genes. In still another embodiment, the protein or peptide is a gene editing protein or peptide that edits or modifies a gene or the genome of the cell.

In one embodiment, the agent is a nucleic acid.

### BRIEF DESCRIPTION OF THE DRAWINGS

FIG. 1A is a schematic illustration of metabolic labeling of cells and subsequent targeting of cell-surface azides using DBCO-agents.

FIG. 1B is a schematic for synthesis of G25 and G400 NP.

FIG. 1C is a schematic for the use of click-functionalized Mannose-acrylate NP for attaching chemical tag to human cell surface.

FIG. 1D is a schematic for biomolecule delivery via molecule conjugation with click-reactive group and then delivery to click-group labeled cells.

FIG. 1E is a bar graph showing labeling and delivery efficiency of human stem cell-derived neural crest-derived cells (hNCC), human embryonic stem cell (hESC), and stem cell-derived mesenchymal stromal cells (hMSC) using azide-containing G400 NP labeling and delivering DBCO-Cy3.

FIGS. 2A-2C illustrate that G400 NP can metabolically label human embryonic stem cells (hESCs) for subsequent targeting of cell-surface azides using DBCO-agents. hESCs were cultured for three (3) days in the absence (control) or presence of 100 μM G400 NP. hESCs were then stained with DBCO-Cy3 at 37° C. for 20 minutes.

FIG. 2A is a representative flow cytometry histogram of ESCs pretreated with PBS and G400 NP for three days and incubated with DBCO-Cy3 for 20 min.

FIG. 2B is a graph showing percentage of Cy3⁺ embryonic stem cells (ESCs) pretreated with PBS and G400 NP for three days and incubated with DBCO-Cy3 for 20 min (n=8).

FIG. 2C is graph showing mean Cy3⁺ fluorescence intensity of ESCs after incubating with sugar materials for three days and detecting with incubating with DBCO-Cy3 for 20 minutes at 37° C.

FIGS. 3A-3C illustrate that G400 NP can metabolically label human neural crest cells (hNCCs) with azido groups, for subsequent targeting of cell-surface azides using DBCO-agents. hNCCs were cultured for three (3) days in the absence (control) or presence of 100 μM G400 NP. hNCCs were then stained with DBCO-Cy3 at 37° C. for 25 minutes.

FIG. 3A is a representative flow cytometry histogram of hNCCs pretreated with PBS and G400 NP for three days and incubated with DBCO-Cy3 for 25 min.

FIG. 3B is a graph showing percentage of Cy3⁺ hNCCs pretreated with PBS and G400 NP for three days and incubated with DBCO-Cy3 for 25 min (n=4).

FIG. 3C is a graph showing mean Cy3⁺ fluorescence intensity of hNCCs pretreated with PBS and G400 NP for three days and incubated with DBCO-Cy3 for 25 min.

FIGS. 4A-4F illustrate that G400 NP can metabolically label human dermal fibroblast (hDF) with azido groups, for subsequent targeting of cell-surface azides DBCO-agents. hDFs were cultured for four (4) days in the absence (control) or presence of 100 μM G400 NP. Negative control: no sugar material or DBCO-agents were added. hDFs were then stained with DBCO-e660/antibody, DBCO-GFP/Cas9, DBCO-Cy3 at 37° C. for 25 minutes.

FIG. 4A is a representative flow cytometry histogram of hDFs pretreated with PBS and G400 NP for four days and incubated with DBCO-e660/antibody for 25 min. FIG. 4B is a graph showing mean e660 fluorescence intensity of hDFs after incubating with sugar materials for four days and detecting with incubating with e660 conjugated antibody coupled to DBCO for 25 minutes at 37° C.

FIG. 4C is a representative flow cytometry histogram of hDFs pretreated with PBS and G400 NP for four days and incubated with DBCO-GFP/Cas9 for 25 min.

FIG. 4D is a graph showing mean GFP fluorescence intensity of hDFs after incubating with sugar materials for four days and detecting with incubating with DBCO-GFP/Cas9 for 25 minutes at 37° C.

FIG. 4E is a representative flow cytometry histogram of hDFs pretreated with PBS and G400 NP for four days and incubated with DBCO-Cy3 for 25 min.

FIG. 4F is a graph showing mean Cy3⁺ fluorescence intensity of hDFs after incubating with sugar materials for four days and detecting with incubating with DBCO-Cy3 for 25 minutes at 37° C.

FIG. 4G is a graph showing flow cytometry and quantification of labeling and protein delivery efficiency to primary adult human dermal fibroblasts using G400 NP, labeling with DBCO-Cy3, and delivery of recombinant Cas9-GFP.

FIGS. 5A and 5B illustrate that G400 NP can metabolically label human dermal fibroblast (hDF) with azido groups, for subsequent targeting of cell-surface azides DBCO-modified Cas9 protein.

FIG. 5A provides confocal images of hDFs after treated with G400 NP for 4 days and incubated with DBCO-GFP/Cas9 for 30 min. Cell nuclei were stained with DAPI. Scale bar: 10 μm.

FIG. 5B provides confocal images of hDFs that are similar to FIG. 5A, except that no G400 NP was added to the cell culture medium.

FIG. 5C provides confocal images showing the delivery of recombinant Cas9-GFP to primary adult human fibroblasts either labeled with G400 NP (top) or unlabeled cells.

FIGS. 6A-6D provides confocal images showing the cytoplasmic delivery of recombinant green fluorescent protein (EGFP) to primary adult human dermal fibroblasts. EGFP was modified with either a DBCO-NHS linker (DBCO-GFP) or a reducible linker DBCO-disulfide-NHS (DBCO-SS-GFP). Endosomes were stained for the marker RabS and showed no colocalization with EGP signal, indicating cytoplasmic delivery. Images show combined DAPI, EGFP, and RabS channels (FIG. 6A), RabS channel (FIG. 6B), EGFP channel (FIG. 6C), and DAPI channel (FIG. 6E).

FIGS. 7A-7F provides confocal images showing the cytoplasmic delivery of recombinant green fluorescent protein (EGFP) to stem cell-derived adipocytes. EGFP was modified with either a DBCO-NHS linker (DBCO-GFP) or a reducible linker DBCO-disulfide-NHS (DBCO-SS-GFP). Lipid droplets were stained with the neutral lipid stain, LipidTox, and showed no colocalization with EGP signal, indicating exclusion from intracellular vesicles, indicating cytoplasmic delivery. Images show combined EGFP and LipidTox channels (FIG. 7A, FIG. 7D), LipidTox channel (FIG. 7B, FIG. 7E), and EGFP channel (FIG. 7C, FIG. 7F).

FIG. 8A is a bar graph showing a timecourse of Cas9-EGFP DBCO modification by measuring loss of primary amine signal from OPA fluorescence reactivity assay relative to unmodified protein.

FIG. 8B is a gel showing cutting activity assay of DBCO-modified Cas9-GFP. Cut fragments of target DNA sequence in agarose electrophoresis gel demonstrate that DBCO-modified Cas9-EGFP retains nuclease activity.

FIG. 9 is a bar graph showing that oligonucleotide-ELISA demonstrates that DBCO-modification of recombinant Oct4 protein at 1:1, 1:5, and 1:10 Oct4:DBCO ratio does not abrogate Oct4 binding to its target promoter sequence.

FIG. 10A provides confocal images showing delivery of AlexaFluor 488-Secondary antibody to culture intact mouse embryos labeled for two days with G400 NP (top) or unlabeled (bottom).

FIG. 10B is a bar graph showing quantification of antibody uptake in cells of embryo by mean and max green fluorescence intensity.

FIG. 10C is a graph showing delivery of Cas9 targeting the Rosa26 locus to culture mouse embryos labeled with G400 NP for 2 days. The left panel shows Rosa26 locus sequence shows five embryos indel percentages, indicating a high degree of cutting efficiency in embryo cells. The right panel shows that of 8 embryos sequenced after Cas9-EGFP delivery, 75% showed direct evidence of active Cas9 after delivery by indels present at the Rosa26 locus.

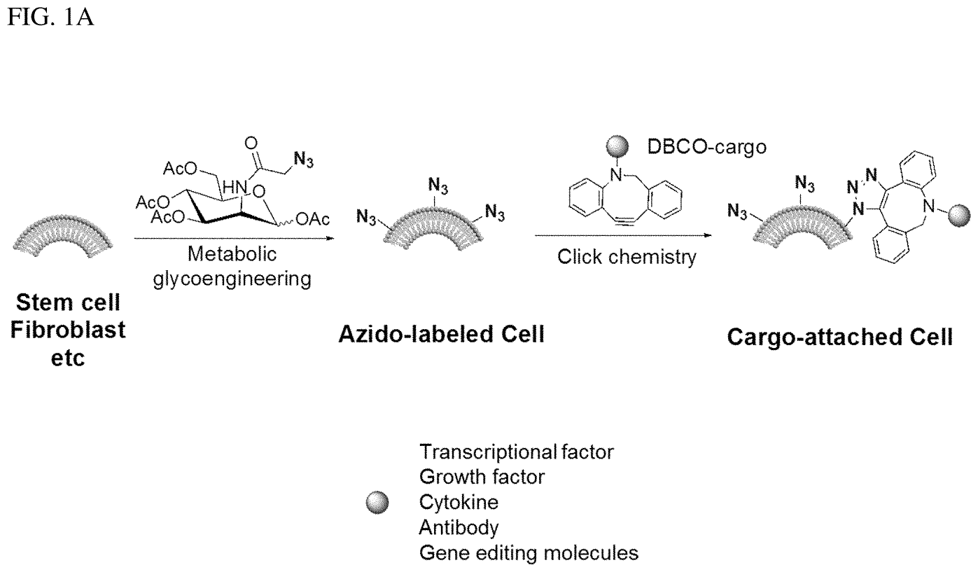

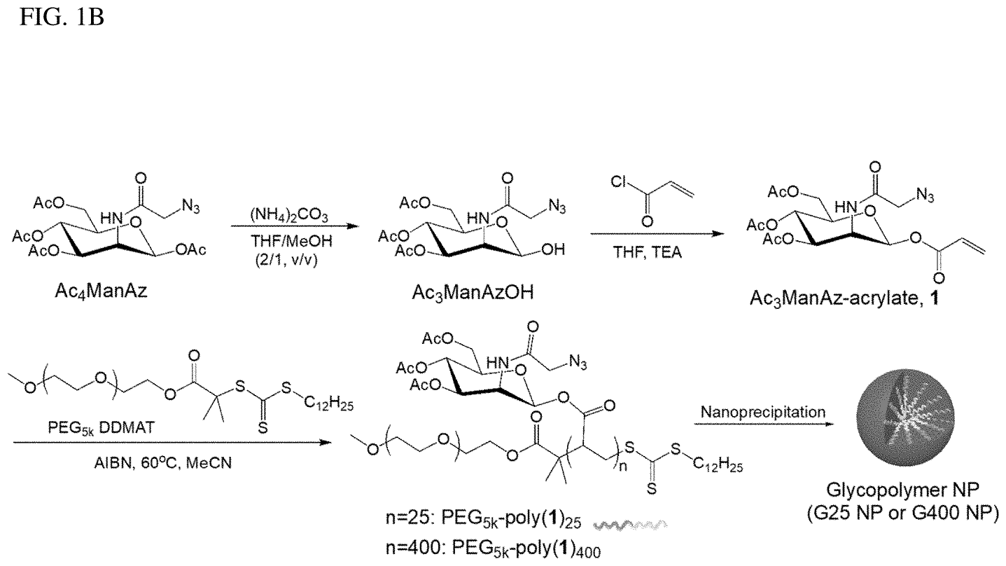

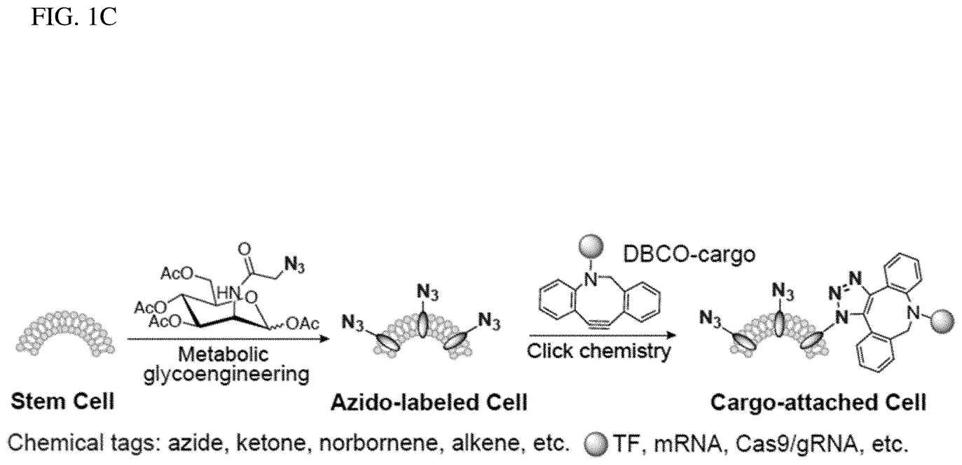

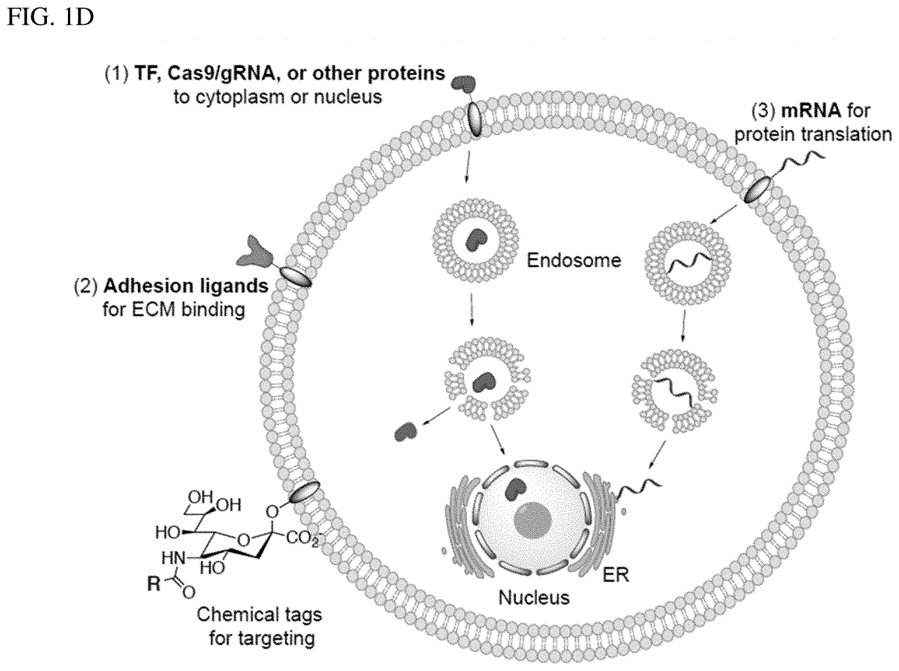

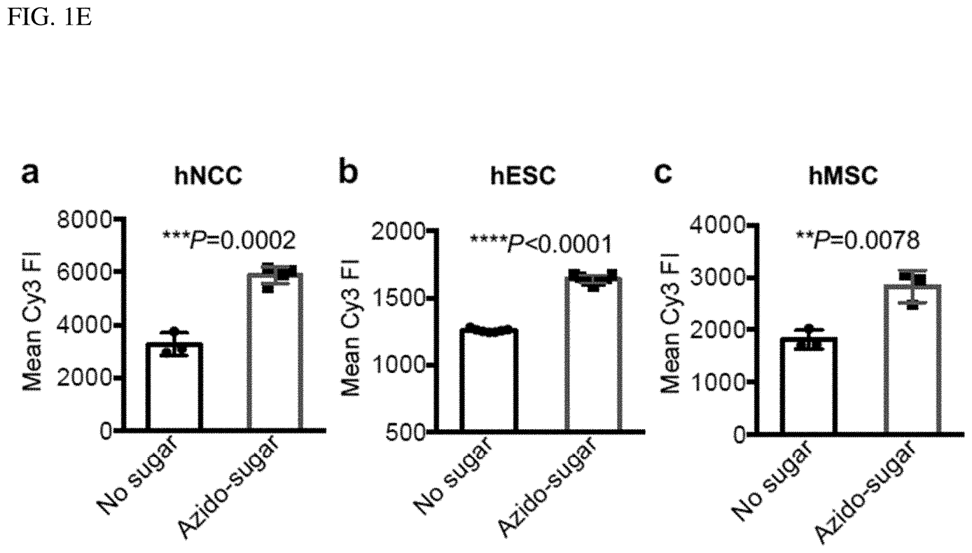

### DETAILED DESCRIPTION OF THE INVENTION

Disclosed herein are compositions and methods for labeling cells using click chemistry reagents. The compositions and methods disclosed herein provide a specific and efficient means of delivering desired agents to a variety of cell types.

### I. DEFINITIONS

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” usually means within 5%, or more preferably within 1%, of a given value or range.

As used herein, the term “subject” includes any subject who may benefit from being administered a hydrogel or an implantable drug delivery device of the invention. The term “subject” includes animals, e.g., vertebrates, amphibians, fish, mammals, non-human animals, including humans and primates, such as chimpanzees, monkeys and the like. In one embodiment of the invention, the subject is a human. The term “subject” also includes agriculturally productive livestock, for example, cattle, sheep, goats, horses, pigs, donkeys, camels, buffalo, rabbits, chickens, turkeys, ducks, geese and bees; and domestic pets, for example, dogs, cats, caged birds and aquarium fish, and also so-called test animals, for example, hamsters, guinea pigs, rats and mice.

As used herein, the term “agent” or “moiety” or “cargo” is defined as any chemical entity that has certain function or activity. An agent or moiety includes, but is not limited to an atom, a chemical group, a small molecule organic compound, an inorganic compound, a nucleoside, a nucleotide, a nucleobase, a sugar, a nucleic acid, an amino acid, a peptide, a polypeptide, a protein, a fusion protein, or a protein complex. The agent or moiety may be detected by methods known in the art. For example, an agent or moiety may be chemiluminescent or fluorescent and can be detected by any suitable chemiluminescent assays known in the art. The function or activity of an agent or moiety may include any physical, chemical, biological, or physiological function or activity. For example, in some embodiments, the agent or moiety may be a radioactive isotope and its activity may include radioactivity. In some other embodiments, the agent or moiety may be enzyme, e.g., nuclease, and its activity or function may include the enzyme activity.

Generally, the term “treatment” or “treating” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating can include suppressing, inhibiting, preventing, treating, or a combination thereof. Treating refers, inter alia, to increasing time to disease progression, expediting remission, inducing remission, augmenting remission, speeding recovery, increasing efficacy of or decreasing resistance to alternative therapeutics, or a combination thereof. “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment, symptoms are secondary. “Primary” refers to a symptom that is a direct result of a disorder, e.g., diabetes, while, secondary refers to a symptom that is derived from or consequent to a primary cause. Symptoms may be any manifestation of a disease or pathological condition.

As used herein, the term “plurality” intends more than one, and may be used interchangeably, in some embodiments, with the term “population.”

In one embodiment, a plurality of cells refers to at least two cells, for example, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 1500, at least 2000, at least 5000, at least 10⁴, at least 10⁵, or more cells. In some embodiments, the plurality of cells are homogenous. In some embodiments, the plurality of cells are heterogenous. In some embodiments, the plurality of cells are primary cells. In some embodiments, the plurality of cells are epithelial cells, fibroblast cells, neuronal cells, endothelial cells, stem cells, and/or immune cells. In some embodiments, the plurality of cells are stem cells, e.g., embryonic stem cells (ESCs), induced pluripotent stem cells, mobilized peripheral blood stem cells, astrocyte, blastocoel, blastocyst, bone marrow stromal cells, cord blood stem cells, hematopoietic stem cells, mesenchymal stem cells, neural stem cells, somatic stem cells, and/or trophoblast stem cells. In some embodiments, the plurality of cells comprise a heterogenous stem cell pool. In some embodiments, the plurality of cells comprise a single-type stem cell pool.

In one embodiment, a plurality of click reagents refers to at least two click reagents, for example, at least 3, at least 4, at least 5, at least 6, at least 7, at least 8, at least 9, at least 10, or more click reagents, e.g., as described herein. In one embodiment, each click reagent in a plurality may be the same or may be different. In some embodiments, each click reagent in a plurality of click reagents may be independently selected from the group consisting of azide, dibenzocyclooctyne (DBCO), transcyclooctene, tetrazine, norbornene, and variants thereof.

In one embodiment, a plurality of click reagents refers to at least two click reagents, for example, a first click reagent and a second click reagent. In one embodiment, a plurality of click reagents refers to at least 3 click reagents, for example, a first click reagent, a second click reagent, and a third click reagent. In one embodiment, a plurality of click reagents refers to at least 4 click reagents, for example, a first click reagent, a second click reagent, a third click reagent, and a fourth click reagent. In one embodiment, a plurality of click reagents refers to at least 5 click reagents, for example, a first click reagent, a second click reagent, a third click reagent, a fourth click reagent, and a fifth click reagent. In one embodiment, a plurality of click reagents refers to at least 6 click reagents, for example, a first click reagent, a second click reagent, a third click reagent, a fourth click reagent, a fifth click reagent, and a sixth click reagent. In one embodiment, a plurality of click reagents refers to at least 7 click reagents, for example, a first click reagent, a second click reagent, a third click reagent, a fourth click reagent, a fifth click reagent, a sixth click reagent, and a seventh click reagent. In one embodiment, a plurality of click reagents refers to at least 8 click reagents, for example, a first click reagent, a second click reagent, a third click reagent, a fourth click reagent, a fifth click reagent, a sixth click reagent, a seventh click reagent, and an eighth click reagent. In one embodiment, a plurality of click reagents refers to at least 9 click reagents, for example, a first click reagent, a second click reagent, a third click reagent, a fourth click reagent, a fifth click reagent, a sixth click reagent, a seventh click reagent, an eighth click reagent, and a ninth click reagent. In one embodiment, a plurality of click reagents refers to at least 10 click reagents, for example, a first click reagent, a second click reagent, a third click reagent, a fourth click reagent, a fifth click reagent, a sixth click reagent, a seventh click reagent, an eighth click reagent, a ninth click reagent, and a tenth click reagent.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%. Accordingly, as used herein, the term “treatment” or “treating” includes any administration of a compound described herein and includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology).

Efficacy of treatment is determined in association with any known method for diagnosing the disorder. Alleviation of one or more symptoms of the disorder indicates that the compound confers a clinical benefit. Any of the therapeutic methods described to above can be applied to any suitable subject including, for example, mammals such as dogs, cats, cows, horses, rabbits, monkeys, and most preferably, humans.

### II. CLICK CHEMISTRY REAGENTS

In one embodiment, the invention features compositions and reagents for labeling cells, e.g., stem cells or primary cells, using click chemistry reagents. Metabolic glycoengineering of unnatural sugars, azido-sugars for example, provides a facile yet powerful way to introduce chemical groups onto the cell surface in the form of glycoproteins. Click-labeled cells can be targeted in vitro or in vivo with agents of interest coupled to a counterpart click moiety. In this manner, virtually any agent can be targeted to cells, and covalently coupled to cell surface glycoproteins, using click chemistry.

In some embodiments, an agent is delivered to a cell intracellularly. In some embodiments, the agent retains its structural integrity, or function or activity after being delivered to a cell intracellularly. For example, a nuclease, e.g., Cas9, retains its nuclease activity after being delivered to a cell intracellularly.

Click Functionalized Polymers

In some examples, the present invention provides a click functionalized polysaccharide polymer which is a product of radical-catalyzed polymerization involving a reaction between one or more saccharide monomers. In this radical-catalyzed polymerization, saccharide monomers are polymerized together to form a polysaccharide polymer. Each saccharide monomer involved in the radical-catalyzed polymerization comprises a saccharide molecule; a click reagent that is attached to the saccharide molecule; and a moiety comprising a functional group amenable to radical polymerization that is attached to the saccharide molecule. The product of the radical-catalyzed polymerization is a click functionalized polysaccharide polymer that comprises repeating saccharide units, in which each saccharide unit is attached, e.g., covalently attached, to a click reagent. The click functionalized polymers are described in PCT Application No. PCT/US2019/051621, the entire contents of which are hereby incorporated herein by reference.

In other examples, the present invention also provides a click-functionalized amphiphilic polymer which is a product of radical-catalyzed polymerization involving a reaction between a reagent comprising a hydrophilic portion and one or more saccharide monomers. In this radical-catalyzed polymerization, saccharide monomers are polymerized together to form a polysaccharide polymer, and the hydrophilic portion becomes attached to the polysaccharide polymer. Each saccharide monomer involved in the radical-catalyzed polymerization comprises a saccharide molecule; a click reagent that is attached to the saccharide molecule; and a moiety comprising a functional group amenable to radical polymerization attached to the saccharide molecule. Thus, in some examples, the product of this radical-catalyzed polymerization is a click functionalized polysaccharide polymer that comprises a hydrophilic portion and repeating saccharide units, and in which each saccharide unit is attached, e.g., covalently attached, to a click reagent.

A polymer of the present invention is introduced into a cell, e.g., as a part of a nanoparticle, the polymer is subjected to hydrolysis, resulting in release inside the cell of individual saccharide monomers attached to a click-reagent. The individual saccharide monomers attached to a click reagent are then subjected to metabolic glycoengineering inside the cell, resulting in incorporation of the saccharide monomers attached to a click reagent into post-translational modifications of, inter alia, proteins of the plasma membrane. The click reagents are then displayed on the cell surface as the proteins span the plasma membrane. As a result, the cell surface becomes labeled with a click reagent.

Any saccharide molecule amenable to metabolic glycoengineering inside a cell may be used to prepare saccharide monomers for preparing click functionalized polymers of the invention. In certain embodiments, the saccharide molecule may be selected from the group consisting of mannose, galactose, fucose and sialic acid. In one specific embodiment, the saccharide molecule may be mannose.

In some examples, in the saccharide monomers, the click reagent may be attached to the saccharide molecule at the C2 position of the sugar moiety. For example, the click reagent may be an azide, and the saccharide molecule may be a mannose, e.g., an acetylated mannose. As illustrated below, an azide may be attached at the C2 position of an acetylated mannose:

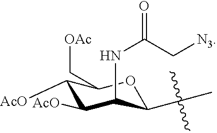

The term “click reagent”, which may be used herein interchangeably with the term “click chemistry reagent” and “click moiety”, refers to a reagent that can rapidly and selectively react (“click”) with its counterpart click reagent under mild conditions in aqueous solution. The mild conditions may include any one of neutral pH, aqueous solution and ambient temperature, with low reactant concentrations. Any suitable click reagent may be used in the context of the present invention. Exemplary click pair reagents are well known to one of skill in the art and include, but are not limited to, moieties that comprise azide and dibenzocyclooctyne (DBCO), tetrazine and transcyclooctene, and tetrazine and norbornene, with the structures illustrated below.

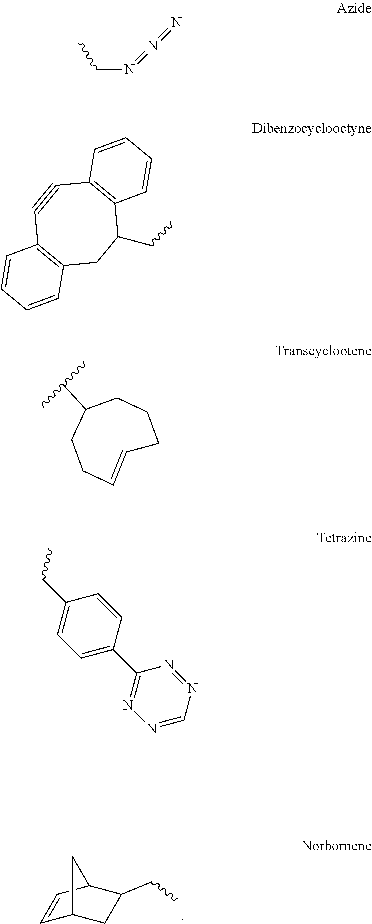

In some embodiments, the click reagent may be an azide. The term “azide” or “azide moiety”, as used herein, includes molecules that comprise an azide moiety as shown above. In some examples, azide may be attached to the saccharide molecule with a suitable spacer moiety. In a specific example, the spacer moiety comprises an aminocarbonyl linkage. The term “aminocarbonyl” or “amide”, as used herein, includes compounds or moieties which contain a nitrogen atom which is bonded to the carbon of a carbonyl or a thiocarbonyl group. This term includes “alkaminocarbonyl” or “alkylaminocarbonyl” groups wherein alkyl, alkenyl, aryl or alkynyl groups are bound to an amino group bound to a carbonyl group. In one specific example, the azide moiety and the spacer moiety may be represented by the following structure:

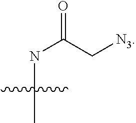 [SMILES: *NC(=O)CN=[N+]=[N-]]

A counterpart click reagent for an azide is dibenzocyclooctyne (DBCO). In some embodiments, the click reagent may be DBCO. As used herein, the term “DBCO” or “DBCO moiety” includes molecules that may comprise a DBCO moiety as shown above. In some examples, DBCO is attached to the saccharide molecule with a suitable spacer moiety, e.g., comprising an aminocarbonyl or an alkylamino linkage. The term “alkylamino”, as used herein, includes moieties wherein a nitrogen atom is covalently bonded to at least one carbon or heteroatom and to at least one alkyl group. This term also includes “dialkylamino”, wherein the nitrogen atom is bound to at least two alkyl groups.

In some embodiments, the click reagent may be tetrazine. As used herein, the term “tetrazine” or “tetrazine moiety” includes molecules that may comprise a tetrazine moiety as shown above. In some examples, transcyclooctene is attached to the saccharide molecule with a suitable spacer moiety, e.g., comprising an aminocarbonyl or an alkylamino linkage. Exemplary tetrazine moieties suitable within the context of the present invention include, but are not limited to, the structures shown below (see, e.g., Karver et al., (2011) Bioconjugate Chem. 22:2263-2270, and WO 2014/065860, the entire contents of each of which are hereby incorporated herein by reference):

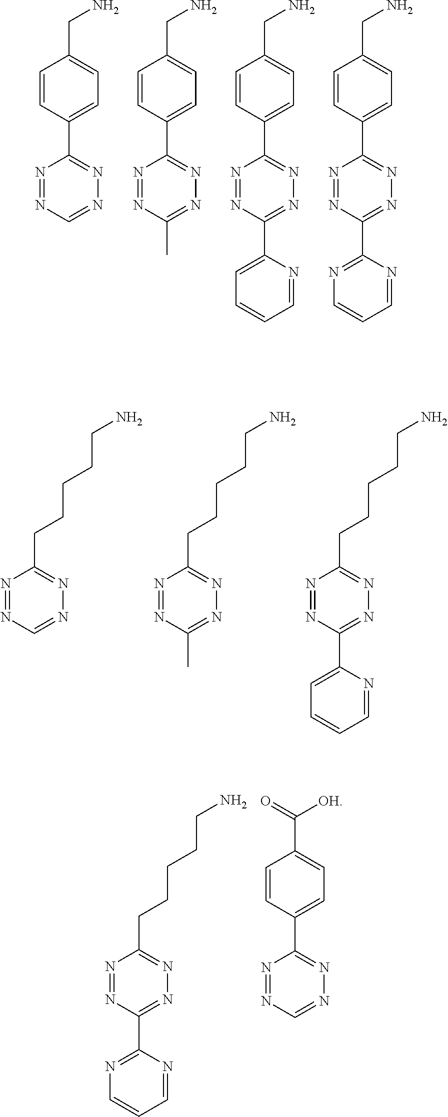

In other examples, exemplary tetrazines that may be used in the context of the present invention are described in U.S. Pat. No. 8,236,949, the entire contents of which are hereby incorporated herein by reference.

One of the counterpart click reagent for a tetrazine is transcyclooctyne. In some embodiments, the click reagent in the context of the present invention may be transcyclooctene. As used herein, the term “transcyclooctene” or “transcyclooctene moiety” includes molecules that may comprise a transcyclooctene moiety as shown above. In some examples, transcyclooctene is attached to the saccharide molecule with a suitable spacer moiety, e.g., comprising an aminocarbonyl or an alkylamino linkage. Exemplary transcyclooctenes that may be used in the context of the present invention include the transyclooctynes described, e.g., in U.S. Pat. No. 8,236,949, the entire contents of which are hereby incorporated herein by reference.

Another counterpart reagent for tetrazine is norbornene (Nb). In some embodiments, the click reagent in the context of the present invention may be norbornene. As used herein, the terms “norbornene” and “norbornene moieties” include but are not limited to the norbornene moiety as shown above, including a moiety comprising norbornadiene and norbornene groups. In some examples, norbornene is attached to the saccharide molecule with a suitable spacer moiety, e.g., comprising an aminocarbonyl or an alkylamino linkage.

In addition to the click reagent, the saccharide monomer may also comprise a moiety comprising a functional group amenable to radical polymerization. The presence of such a moiety in the saccharide monomer provides the means to polymerize the saccharide moieties, thereby forming a click functionalized polymer of the invention. The moiety comprising a functional group amenable to radical polymerization may comprise a double bond. For example, the moiety comprising a functional group amenable to radical polymerization may comprise an acrylate or a methacrylate. In one specific example, the moiety comprising a functional group amenable to radical polymerization comprises an acrylate. In another specific example, the moiety comprising a functional group amenable to radical polymerization comprises a methacrylate.

The moiety comprising a functional group amenable to radical polymerization may be attached to the saccharide molecule, e.g., mannose, galactose, fucose or sialic acid, at the C1 position, the C3 position, the C4 position or the C5 position of the saccharide molecule. In one specific embodiment, the moiety comprising a functional group amenable to radical polymerization is attached to the saccharide molecule at the C1 position.

Illustrated below is an exemplary saccharide monomer comprising mannose as the saccharide molecule, an azide as the click reagent attached at the C2 position of the mannose and the acrylate as the moiety comprising a functional group amenable to radical polymerization attached at the C1 position. The exemplary saccharide monomer is further acetylated at the C3, C4 and C5 positions of the mannose:

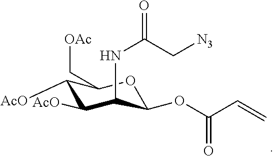

The saccharide monomer used in the radical-catalyzed polymerization to produce the polymers of the present invention may further comprise one or more hydrolysable substituents at any position that is not occupied by the click reagent or moiety comprising a functional group amenable to radical polymerization. For example, a hydrolysable substituent may be present at the C1 position, the C3 position, the C4 position or C5 position of the saccharide monomer. In some examples, the hydrolysable substituent contributes to the hydrophobicity of the polymer, but, once inside the cell, may be hydrolyzed and converted to a hydroxyl group. In some example, the hydrolysable substituent is represented by formula (1):

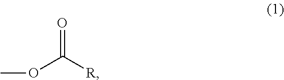

wherein R is alkyl. In a specific example, R is methyl.

The term “alkyl”, as used herein, includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (e.g., isopropyl, tert-butyl, isobutyl, etc.). The term alkyl also includes alkyl groups which can further include oxygen, nitrogen, sulfur or phosphorous atoms replacing one or more carbons of the hydrocarbon backbone. In some examples, a straight chain or branched chain alkyl may have 6 or fewer carbon atoms in its backbone (e.g., C₁-C₆ for straight chain, C₃-C₆ for branched chain), and more preferably 4 or fewer. The term “C₁-C₆” includes alkyl groups containing 1 to 6 carbon atoms.

In some examples, the click functionalized polysaccharide polymers of the present invention may comprise 10 to 1000 saccharide units, i.e., 10 to 1000 saccharide monomers attached together to form the click functionalized polysaccharide polymer. For example, the polymers of the invention may comprise 20 to 500, 100 to 500 or 200 to 600 saccharide units. In one specific example, the polymer of the invention may comprise 10-50 saccharide units, e.g., 25 saccharide units. In another specific example, the polymer of the invention may comprise 300-500 saccharide units, e.g., 400 saccharide units. In one specific embodiment, the polymer of the invention may comprise the structure of formula (2):

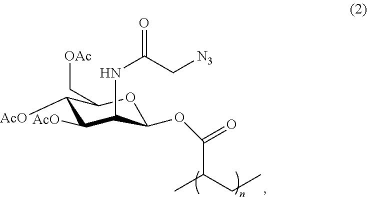

wherein n is a number between 10 and 1000.

In some examples, the click functionalized polysaccharide polymer of the present invention may further comprise a hydrophilic portion. The hydrophilic portion may be attached to the repeating saccharide units in which each saccharide unit is attached, e.g., covalently attached, to a click reagent. The hydrophilic portion may comprise a hydrophilic polymer, such as polyethylene oxide (PEG). In some examples, the PEG may comprise between 20 and 450 PEG units, e.g., about 100 to about 150 PEG units. In some examples, the PEG may have an average molecular weight of about 500 to about 20,000 Daltons, e.g., about 2,000 and about 10,000 Dalton. In one example, the PEG has an average molecular weight of about 5,000 Daltons.

In some examples, the click functionalized polysaccharide polymer of the invention comprising a hydrophilic portion may comprise the structure of formula (3):

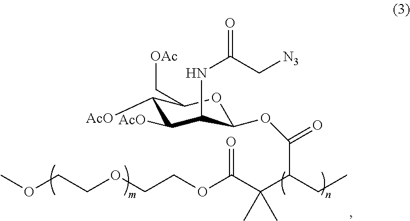

wherein n is a number between 10 and 1000; and m is a number between 45 and 200.

The polymers of the invention are produced by subjecting saccharide monomers as described above and, optionally, the hydrophilic portion, to a radical-catalyzed polymerization. In some examples, the radical-catalyzed polymerization may be reversible addition-fragmentation chain transfer (RAFT) polymerization. The RAFT polymerization involves conventional free radical polymerization of a substituted monomer in the presence of a suitable chain transfer agent (RAFT agent or CTA), which mediate the polymerization via a reversible chain-transfer process.

Any suitable RAFT reagent may be used in the context of the present invention. Exemplary RAFT agents may be found, e.g., in the SIGMA-ALDRICH catalog and may comprise a thiocarbonate moiety, a dithiocarbamate moiety or a dithiobenzoate moiety. In one specific example, the RAFT agent may comprise a thiocarbonate moiety, e.g., 2-(dodecylthiocarbonothioylthio)-2-methylpropionate.

In another specific example, the RAFT agent may comprise poly(ethylene glycol) methyl ether 2-(dodecylthiocarbonothioylthio)-2-methylpropionate. In this example, when the RAFT agent participates in the radical-catalyzed polymerization, the poly(ethylene glycol) portion of the RAFT agent becomes attached to the resulting click functionalized polysaccharide polymer and becomes the hydrophilic portion of the polymer. An exemplary product of the RAFT polymerization that comprises the use of poly(ethylene glycol) methyl ether 2-(dodecylthiocarbonothioylthio)-2-methylpropionate as the RAFT agent is the structure of formula (4):

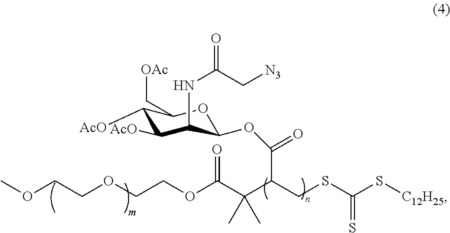

wherein n is a number between 10 and 1000; and m is a number between 45 and 200.

Nanoparticles

The present invention also provides nanoparticles for labeling cells with a click reagent. The nanoparticles may comprise the click functionalized polysaccharide polymer of the invention as described above.

In some examples, the nanoparticle may be self-assembling, i.e., may spontaneously form when click functionalized polysaccharide polymer of the invention, once prepared, is exposed to certain conditions, such as an aqueous solvent or a physiological pH, or when the click functionalized polysaccharide polymer of the invention is subjected to nanoprecipitation. Scheme 1 below illustrates preparation of an exemplary nanoparticle of the invention starting from synthesis of a click functionalized polysaccharide polymer using RAFT polymerization. The RAFT reagent used in the RAFT polymerization is poly(ethylene glycol) methyl ether 2-(dodecylthiocarbonothioylthio)-2-methylpropionate. The saccharide monomer used in the RAFT polymerization to produce the click functionalized polysaccharide polymer is Ac₃ManAz-acrylate. The Ac₃ManAz-acrylate comprises mannose as the saccharide molecule which is functionalized at the C1 position with an azide as the click reagent and at the C2 positon with an acrylate as the moiety comprising a functional group amenable to radical polymerization. The Ac₃ManAz-acrylate further comprises acetyl groups at the C3, C4 and C5 positions as the hydrolysable substituents. The resulting polymer also comprises PEG₅ₖ (or PEG having an average molecular weight of about 5000 Daltons) as the hydrophilic portion. In the last step, a nanoparticle is produced by subjecting the click functionalized polysaccharide polymer of the invention to nanoprecipitation.

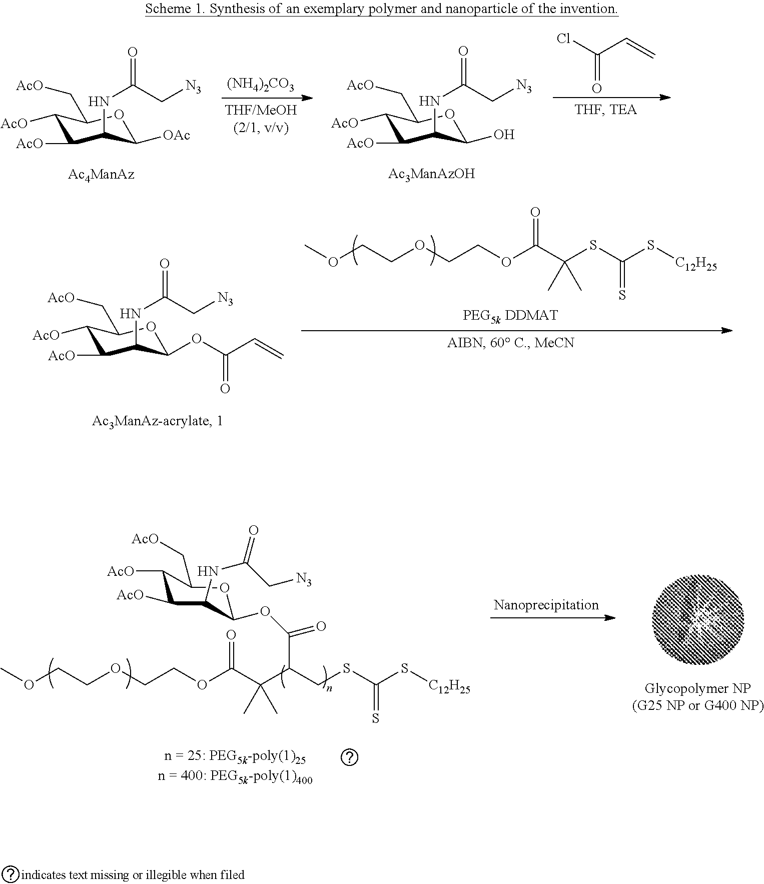

In other examples, the nanoparticle of the invention does not comprise a click functionalized polysaccharide polymer. Rather, the nanoparticle of the invention may comprise a saccharide molecule, e.g., a monomeric saccharide molecule, attached to a click reagent. For example, the saccharide molecule may be selected from the group consisting of mannose, galactose, fucose and sialic acid. In one specific example, the saccharide molecule is mannose.

The click reagent may be attached to the saccharide molecule at the C2 position and may comprise any of the click reagents as described above for saccharide monomers. The saccharide molecule may also comprise one or more hydrolysable substituents at the C1, C3, C4 and/or C5 positions of the saccharide molecule as described above for saccharide monomers.

The nanoparticle useful in the context of the present invention may be selected from the group consisting of a carbon-based nanoparticle, a ceramic nanoparticle, a metal nanoparticle, a semiconductor nanoparticle, a polymeric nanoparticle and a lipid-based nanoparticle. In one specific example, the nanoparticle may be a lipid-based nanoparticle, e.g., a liposome or a micelle. In another specific example, the nanoparticle useful in the context of the present invention may be a semiconductor nanoparticle, e.g., a silica nanoparticle.

### III. COMPOSITIONS AND METHODS FOR LABELING CELLS

In some embodiments, the present invention also provides a method for labeling a cell with a click reagent that comprises contacting the cell with the click functionalized polysaccharide polymer of the invention as described above. In other embodiments, the present invention also provides a method for labeling a cell with a click reagent that comprises contacting the cell with a nanoparticle of the invention as described above. Contacting the cell with the polymers or nanoparticles of the invention can take place in vitro, ex vivo, or in vivo. The compositions and methods for labeling cells have been described in detail in PCT/US2019/051621, the entire contents of which are incorporated herein by reference.

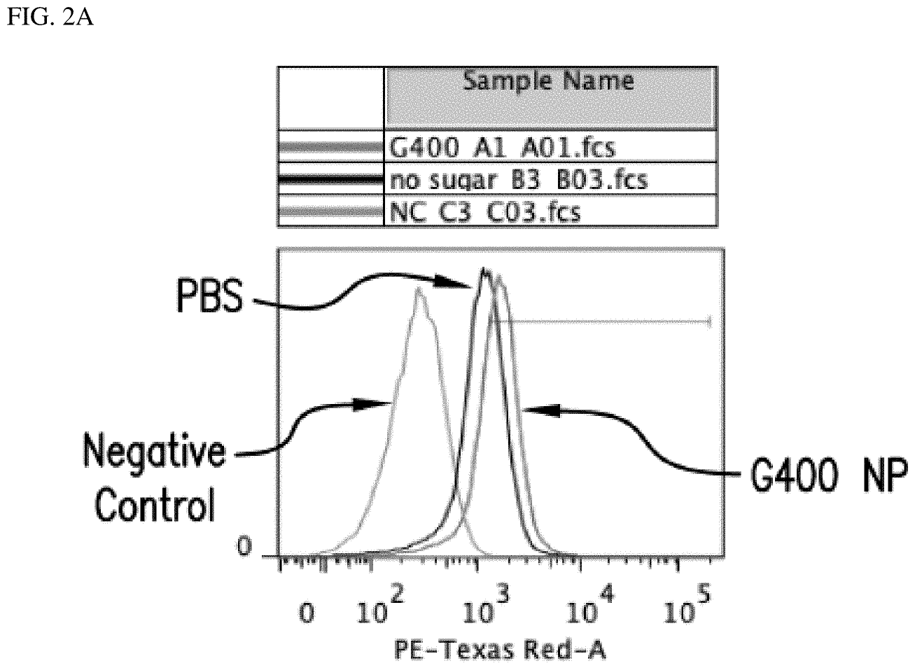

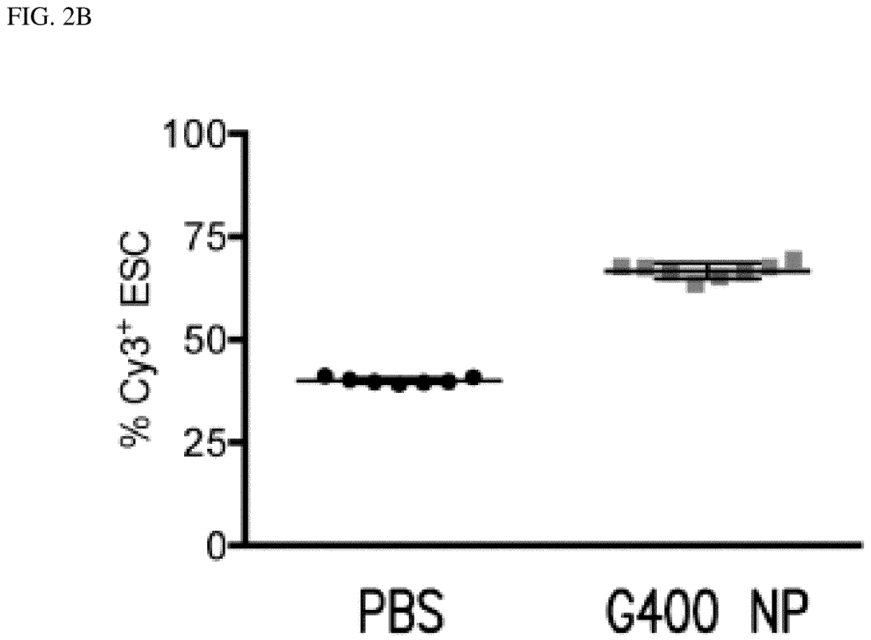

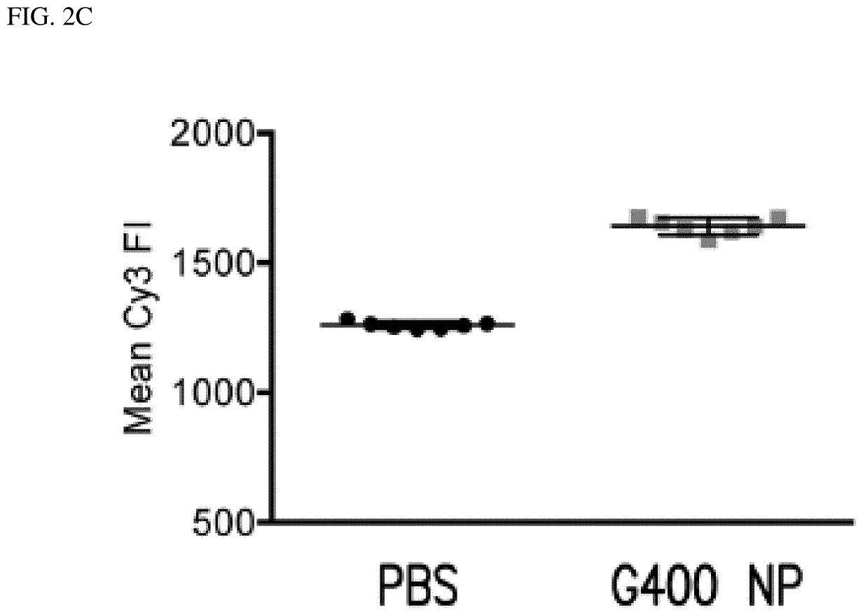

The foregoing polymer and nanoparticle compositions can be used to metabolically label the surface of cells with click chemistry reagents. Click chemistry reagents including sugar moieties, and nanoparticles comprising the click chemistry reagents as described herein, can enter cells by endocytosis, and subsequently disassemble and degrade by hydrolysis or enzymatic degradation. The released sugar-click reagent is metabolically processed, and is presented on the surface of the cell in the form of a glycoprotein. This process is illustrated schematically for the exemplary embodiment of azido-sugar nanoparticles in FIGS. 2E and 2F of PCT/US2019/051621.

Preferably, cells are contacted with an effective amount of the click chemistry reagent. In some embodiments, the effective amount is an amount sufficient to metabolically label at least 10% of cell surface glycoproteins with a click moiety, e.g., an azide moiety, a DBCO moiety, a transcyclooctene moiety, a tetrazine moiety, or a norbornene moiety. The amount of a click chemistry reagent needed to metabolically label cells can readily be determined for each reagent and each cell type. In exemplary embodiments, the click reagent is provided to cells at a concentration of 1 nM to 1 μM. In other exemplary embodiments, the click reagent is provided to cells at a concentration of 1 μM to 1 mM. In other exemplary embodiments, the click reagent is provided to cells at a concentration of 1 mM to 1 M.

In some embodiments, the amount of the chemistry reagent is sufficient to label about 5% to about 100% of the cell surface glycoproteins with the click moiety. In certain embodiments, the amount of the chemistry reagent is sufficient to label about 5%, about 10%, about 20%, about 30%, about 40%, about 50%, about 60%, about 70%, about 80%, about 90%, or about 99% of the cell surface glycoproteins. The intermediaries and ranges between the recited values are contemplated as part of the invention. The labeling efficiency can be readily determined in any methods known in the art. For example, the labeling efficiency can be determined by measuring the percentage of the total cell surface glycoproteins that can react to the counter click reagent using any suitable fluorescence assays and/or immunoassays.

In certain embodiments, at least about 10⁷ to about 10⁸ click moieties, e.g., azide, are used to label a single cell. In some embodiments, about 1×10⁷, about 2×10⁷, about 3×10⁷, about 4×10⁷, about 5×10⁷, about 6×10⁷, about 7×10⁷, about 8×10⁷, about 9×10⁷, or about 10⁸ click moieties, or more than about 10⁸ click moieties are used to label a single cell. Intermediaries and ranges between the recited values are contemplated as part of the invention.

Virtually any cell type can be labeled with a click reagent in this manner. For example, this method can be used to label an epithelial cell, a fibroblast cell, a neuronal cell, an endothelial cell, a stem cell, and/or an immune cell with a click reagent. In some exemplary embodiments, the click chemistry reagents disclosed herein can be used to label leukocytes, e.g. peripheral blood leukocytes, spleen leukocytes, lymph node leukocytes, hybridoma cells, T cells (cytotoxic/suppressor, helper, memory, naive, and primed), B cells (memory and naive), monocytes, macrophages, granulocytes (basophils, eosinophils, and neutrophils), natural killer cells, natural suppressor cells, thymocytes, and dendritic cells; cells of the hematopoietic system, e.g. hematopoietic stem cells (CD34+), proerythroblasts, normoblasts, promyelocytes, reticulocytes, erythrocytes, pre-erythrocytes, myeloblasts, erythroblasts, megakaryocytes, B cell progenitors, T cell progenitors, thymocytes, macrophages, mast cells, and thrombocytes; stromal cells, e.g. adipocytes, fibroblasts, adventitial reticular cells, endothelial cells, undifferentiated mesenchymal cells, epithelial cells including squamous, cuboid, columnar, squamous keratinized, and squamous non-keratinized cells, and pericytes; cells of the skeleton and musculature, e.g. myocytes (heart, striated, and smooth), osteoblasts, osteoclasts, osteocytes, synoviocytes, chondroblasts, chondrocytes, endochondral fibroblasts, and perichonondrial fibroblasts; cells of the neural system, e.g., neural crest cells, astrocytes (protoplasmic and fibrous), microglia, oligodendrocytes, and neurons; cells of the digestive tract, e.g. parietal, zymogenic, argentaffin cells of the duodenum, polypeptide-producing endocrine cells (APUD), islets of langerhans (alpha, beta, and delta), hepatocytes, and kupfer cells; cells of the skin, e.g. keratinocytes, langerhans, and melanocytes; cells of the pituitary and hypothalamus, e.g. somatotropic, mammotropic, gonadotropic, thyrotropic, corticotropin, and melanotropic cells; cells of the adrenals and other endocrine glands, e.g. thyroid cells (C cells and epithelial cells); adrenal cells; cells of the reproductive system, e.g. oocytes, spermatozoa, leydig cells, embryonic stem cells, amniocytes, blastocysts, morulas, and zygotes; and tumor cells.

In other exemplary embodiments, the click chemistry reagents disclosed herein are used to label stem cells, e.g., embryonic stem cells (ESCs), induced pluripotent stem cells, mobilized peripheral blood stem cells, astrocyte, blastocoel, blastocyst, bone marrow stromal cells, cord blood stem cells, hematopoietic stem cells, mesenchymal stem cells, neural stem cells, somatic stem cells, and/or trophoblast stem cells.

In certain exemplary embodiments, the click chemistry reagents disclosed herein are used to label fibroblast cells, e.g., dermal fibroblasts, lung fibroblasts, bladder fibroblasts, uterine fibroblasts, vas deferens fibroblasts, tendon fibroblasts, ligament fibroblasts, synovial fibroblasts, foreskin fibroblasts, and cardiac fibroblasts.

In an exemplary embodiment, the cells are contacted with the reagent for a period of time sufficient for cells to take up the reagent by endocytosis. The period of time sufficient for the cell to take up the click chemistry reagent can be determined empirically, for example, by microscopy, flow cytometry, and other standard techniques. In exemplary embodiments, the period of time sufficient for the cell to take up the click chemistry reagent is about 3 hours, about 6 hours, about 12 hours, about 24 hours, about 48 hours, about 72 hours, about 96 hours, about 120 hours, or more. In other embodiments, the period of time sufficient for the cell to take up the click chemistry reagent is about 24-120 hours, about 48-96 hours, or about 48-72 hours. Intermediaries and ranges between the recited values are contemplated as part of the invention.

Metabolic processing of the click chemistry reagent occurs inside the cell, whereby the sugar moiety is partially degraded and incorporated into glycoproteins, which are then displayed on the cell surface. After processing, the cells contain cell surface proteins which comprise carbohydrate molecules labeled with the click moiety.

Accordingly, in another aspect, the invention provides a cell comprising a cell surface glycoprotein, wherein the glycoprotein comprising a carbohydrate covalently linked to a click reagent. In exemplary embodiments, the click reagent comprises azide, dibenzocyclooctyne (DBCO), transcyclooctene, tetrazine and/or norbornene, or variants thereof. In some embodiments, the cell is an isolated cell. In some embodiments, the cell is an epithelial cell, a fibroblast cell, a neuronal cell, an endothelial cell, or an immune cell. In some exemplary embodiments, the cell is a cell type selected from leukocytes, e.g. peripheral blood leukocytes, spleen leukocytes, lymph node leukocytes, hybridoma cells, T cells (cytotoxic/suppressor, helper, memory, naive, and primed), B cells (memory and naive), monocytes, macrophages, granulocytes (basophils, eosinophils, and neutrophils), natural killer cells, natural suppressor cells, thymocytes, and dendritic cells; cells of the hematopoietic system, e.g. hematopoietic stem cells (CD34+), proerythroblasts, normoblasts, promyelocytes, reticulocytes, erythrocytes, pre-erythrocytes, myeloblasts, erythroblasts, megakaryocytes, B cell progenitors, T cell progenitors, thymocytes, macrophages, mast cells, and thrombocytes; stromal cells, e.g. adipocytes, fibroblasts, adventitial reticular cells, endothelial cells, undifferentiated mesenchymal cells, epithelial cells including squamous, cuboid, columnar, squamous keratinized, and squamous non-keratinized cells, and pericytes; cells of the skeleton and musculature, e.g. myocytes (heart, striated, and smooth), osteoblasts, osteoclasts, osteocytes, synoviocytes, chondroblasts, chondrocytes, endochondral fibroblasts, and perichonondrial fibroblasts; cells of the neural system, e.g. neural crest cells, astrocytes (protoplasmic and fibrous), microglia, oligodendrocytes, and neurons; cells of the digestive tract, e.g. parietal, zymogenic, argentaffin cells of the duodenum, polypeptide-producing endocrine cells (APUD), islets of langerhans (alpha, beta, and delta), hepatocytes, and kupfer cells; cells of the skin, e.g. keratinocytes, langerhans, and melanocytes; cells of the pituitary and hypothalamus, e.g. somatotropic, mammotropic, gonadotropic, thyrotropic, corticotropin, and melanotropic cells; cells of the adrenals and other endocrine glands, e.g. thyroid cells (C cells and epithelial cells); adrenal cells; cells of the reproductive system, e.g. oocytes, spermatozoa, leydig cells, embryonic stem cells, amniocytes, blastocysts, morulas, and zygotes; and tumor cells.

In other exemplary embodiments, the cells are stem cells, e.g., embryonic stem cells (ESCs), induced pluripotent stem cells, mobilized peripheral blood stem cells, astrocyte, blastocoel, blastocyst, bone marrow stromal cells, cord blood stem cells, hematopoietic stem cells, mesenchymal stem cells, neural stem cells, somatic stem cells, and/or trophoblast stem cells.

In certain exemplary embodiments, the cells are fibroblast cells, e.g., dermal fibroblasts, lung fibroblasts, bladder fibroblasts, uterine fibroblasts, vas deferens fibroblasts, tendon fibroblasts, ligament fibroblasts, synovial fibroblasts, foreskin fibroblasts, and cardiac fibroblasts.

The click-labeled cells disclosed herein can, in some embodiments, be administered to a subject, e.g., a mammalian subject, such as a murine subject, a primate subject, or a human subject. In some embodiments, click-labeled cells are administered to a subject as part of a treatment regimen. For example, in some embodiments, click-labeled fibroblast cells can be administered to a subject to introduce a recombinant gene product, such as a protein.

In some aspects, the invention provides compositions and methods for labeling cells with a click reagent of the invention in vivo. In some embodiments, the invention provides a method of labeling cells with a click reagent in vivo, comprising administering a click reagent disclosed herein to a subject. In exemplary embodiments, the click reagent is provided as a polymer, or as a nanoparticle, as described herein. In some embodiments, the click reagent, polymer, or nanoparticle can be incorporated into a polymer scaffold device.

Accordingly, in one aspect, the invention provides a device comprising a polymer scaffold and a click reagent. A number of biomaterial scaffolds are available that allow the migration of cells into an out of the scaffold in vivo. Incorporation of the click reagents of the invention into such scaffolds provides a platform for contacting cells in vivo with the click reagents, thereby allowing metabolic labeling of cells that contact the scaffold in vivo. Labeling of specific cell types in vivo can be achieved by modifying the device to promote recruitment of the desired cells to the scaffold. For example, the device can contain chemoattractants that promote recruitment of specific cell types to the scaffold in vivo. In some embodiments, the click reagents of the invention are formatted as a polymer, e.g., a click functionalized polysaccharide polymer, or as a nanoparticle, as described herein. The device scaffolds suitable for metabolically labeling cells are described in PCT/US2019/051621, incorporated herein by reference.

In some embodiments, cells are labeled with a click reagent in vitro and administered to a subject in the absence of a scaffold. The agent coupled to a counterpart click reagent is administered to the subject separately or together with the click-agent labeled cell. The click reaction between the click reagents allows the agent to be specifically or selectively targeted to the click-reagent labeled cell.

### IV. COMPOSITIONS AND METHODS FOR TARGETING AGENTS TO CELLS USING CLICK CHEMISTRY PAIRS

Cells labeled with click reagents in vitro, ex vivo, or in vivo can be covalently coupled to a moiety of interest using click chemistry. For example, the cell can be contacted with a counterpart click reagent that is, in turn, attached to a moiety, thereby conjugating the moiety to the cell. The contacting can occur in vitro, ex vivo, or in vivo. Accordingly, in one embodiment, cells are labeled with a click reagent in vitro or ex vivo, and are contacted in vitro or ex vivo with a counterpart click reagent that is attached to a moiety for conjugation to the cells. In another embodiment, cells are labeled with a click reagent in vitro or ex vivo, and are contacted in vivo with a counterpart click reagent that is attached to a moiety for conjugation to the cells. In this embodiment, the contacting can be performed by administration of the counterpart click reagent attached to the moiety to a subject who comprises the click-labeled cells. In another embodiment, cells are labeled with a click reagent in vivo, and are contacted in vivo with a counterpart click reagent that is attached to a moiety for conjugation to the cells. Exemplary moieties that can be conjugated to cells in this manner are described below.

Accordingly, the present invention provides cells that include a glycoprotein-agent complex. The glycoprotein-agent complex is formed through specific or selective click reaction between a cell labeled with a click reagent and an agent coupled to a counterpart click reagent. In some embodiments, the glycoprotein-agent complex is located within the cell. In certain embodiments, the agent retains its structural integrity, function, and/or activity while residing within the cell.

In certain embodiments, the present invention provides methods to label and target a cell directly. For example, a cell may be labeled with a click reagent by contacting the cell directly with the click reagent, e.g., G 400 NP. The cell labeled with a click reagent may be subsequently targeted by a counterpart click reagent, e.g., a moiety coupled to DBCO, by a direct contact with the counterpart click reagent. The advantage of direct labeling and/or targeting methods according to this disclosure includes, but is not limited to, that it does not require an intermediary, e.g., exosome.

In some embodiments, the click reagent presented on the surface of a cell, e.g., coupled to a cell surface glycoprotein, may react with its counterpart click reagent that is, in turn, attached to a moiety, thereby conjugating the moiety to the cell. Any moiety may be conjugated to the click labeled cells of the invention using the click reagents. The moiety should be coupled to a click reagent that can rapidly and selectively react (“click”) with its counterpart click reagent, i.e., the click reagent presented on the surface of a cell to be targeted, under mild conditions in aqueous solution. The mild conditions include neutral pH, aqueous solution and ambient temperature, with low reactant concentrations. Exemplary click reagent pairs are well known to one of skill in the art and include, but are not limited to, azide and dibenzocyclooctyne (DBCO), tetrazine and transcyclooctene, and tetrazine and norbornene. Accordingly, a cell labeled with azide can be conjugated to a moiety that is coupled to DBCO. In other embodiments, a cell labeled with DBCO can be conjugated to a moiety that is coupled to azide. In other embodiments, a cell labeled with tetrazine can be conjugated to a moiety that is coupled to transcyclooctene or norbornene. In other embodiments, a cell labeled with transcyclooctene or norbornene can be conjugated to a moiety that is coupled to tetrazine.

In exemplary embodiments, the period of time sufficient for the click labeled cell to be targeted by the moiety is about 5 minutes, about 10 minutes, about 20 minutes, about 30 minutes, about 40 minutes, or more than about 40 minutes. Intermediaries and ranges between recited values are contemplated as part of the invention.

The moiety to be conjugated to the click labeled cells may be of various sizes. In some embodiments, the moiety may be a small protein or nucleic acid and have a molecular weight that is smaller than 10,000 Dalton with a hydrodynamic diameter between about 10 nm and about a thousand nanometer. In some embodiments, the moiety may have a molecular weight of about 1,000 Dalton, about 2,000 Dalton, about 3,000 Dalton, about 4,000 Dalton, about 5,000 Dalton, about 6,000 Dalton, about 7,000 Dalton, about 8,000 Dalton, about 9,000 Dalton, or about 10,000 Dalton. In certain embodiments, the moiety may have a hydrodynamic diameter of about 10 nm, about 20 nm, about 50 nm, about 100 nm, about 200 nm, about 300 nm, about 400 nm, about 500 nm, about 600 nm, about 700 nm, about 800 nm, about 900 nm, or about 1,000 nm. In some embodiment, the moiety may be of middle size, e.g., having a molecular weight between about 10,000 Dalton and about 1 million Dalton. In certain embodiments, the moiety has a molecular weight of about 10,000 Dalton, about 20,000 Dalton, about 50,000 Dalton, about 100,000 Dalton, about 200,000 Dalton, about 300,000 Dalton, about 400,000 Dalton, about 500,000 Dalton, about 600,000 Dalton, about 700,000 Dalton, about 800,000 Dalton, about 900,000 Dalton, or about 1 million Dalton. In certain embodiments, the moiety may also include large size nucleic acid, protein, or complex that contains multiple proteins and/or nucleic acids. Such a moiety may have a molecular weight in the range between about 1 million Dalton and about 1 billion Dalton and have a hydrodynamic diameter that is larger than 1 micrometer. In some embodiments, the moiety may have a molecular weight of about 1 million Dalton, about 2 million Dalton, about 5 million Dalton, about 10 million Dalton, about 20 million Dalton, about 50 million Dalton, about 100 million Dalton, about 200 million Dalton, about 300 million Dalton, about 400 million Dalton, about 500 million Dalton, about 600 million Dalton, about 700 million Dalton, about 800 million Dalton, about 900 million Dalton, or about 1 billion Dalton. In still some other embodiment, the moiety may have a hydrodynamic diameter of greater than about 1 μm, greater than about 2 μm, greater than about 5 μm, greater than about 10 μm, greater than about 20 μm, greater than about 30 μm, greater than about 40 μm, greater than about 50 μm, greater than about 60 μm, greater than about 70 μm, greater than about 80 μm, greater than about 90 μm, or greater than about 100 μm. The intermediaries and ranges between the recited values are contemplated as part of this invention.

Non-limiting examples of moieties that can be targeted to click-labeled cells include a small organic molecule, a small inorganic molecule; a saccharine; a monosaccharide; a disaccharide; a trisaccharide; an oligosaccharide; a polysaccharide; a peptide; a protein, a peptide analog, a peptide derivative; a peptidomimetic; an antibody (polyclonal or monoclonal); an antigen binding fragment of an antibody; a nucleic acid, e.g., an oligonucleotide, an antisense oligonucleotide, siRNAs, shRNAs, a ribozyme, an aptamer, microRNAs, pre-microRNAs, iRNAs, plasmid DNA (e.g. a condensed plasmid DNA), a modified RNA, and a nucleic acid analog or derivative. In some embodiments, the moiety is a therapeutic agent. In other embodiments, the moiety is a detection agent.

In some embodiments, the moieties targeted to a cell are attached, e.g., covalently linked, to a surface glycoprotein and remain on cell surface. In some embodiments, the moieties targeted to a cell are attached, e.g., covalently linked to a surface glycoprotein and are delivered intracellularly. Without wishing to be bound by any theory, the moiety-glycoprotein protein complex formed through click chemistry is engulfed into the cell through endocytosis. After endocytosis, the moiety may be dissociated from the moiety-glycoprotein complex. In certain embodiments, the moiety is coupled to the click reagent through a linker, which can be cleaved within the cell selectively or specifically to facilitate the disassociation of the moiety from the glycoprotein. Exemplary linkers include, but are not limited to disulfide bond, hydrozone bond, or enzyme cleavable bond. The linker can be cleaved in endosome, facilitating the gene-editing moiety to be released from endosome. In some embodiments, the moiety retains its structural integrity, function, or activity after being targeted to a cell. A moiety retains its structural integrity when the moiety retains its intact structure or undergoes some structural changes but retains its function or activity. For example, a nuclease, e.g., Cas9, retains its structural integrity in that although it may undergo some structural change, e.g., partial degradation, it retains its nuclease activity. This allows for the delivery of an agent to a cell intracellularly so as to modify or manipulate the cell, e.g., physically, chemically, biochemically, physiologically, genetically, or epigenetically.

This strategy allows cells in vivo, ex vivo, or in vitro to be covalently coupled to virtually any agent. In some embodiments, an agent is targeted to a cell in vitro, comprising contacting a cell coupled to a click reagent with an agent coupled to a counterpart click reagent. The cell may be cultured in vitro. In general, the moiety coupled to a click reagent may be added to a cell culture medium to contact the cell coupled to a counterpart click reagent.

In some embodiments, a cell may be contacted in vivo, ex vivo, or in vitro with at least one, at least two, at least three, at least four, at least 5, at least 6, at least 7, at least 8, at least 9, or at least 10, or more agents coupled to a click reagent, optionally, a plurality of times.

In some embodiments, a plurality of cells may be contacted in vivo, ex vivo, or in vitro with at least one, at least two, at least three, at least four, at least 5, at least 6, at least 7, at least 8, at least 9, or at least 10, or more agents coupled to a click reagent, optionally, a plurality of times.

Accordingly, the present invention provides a cell comprising an agent/moiety attached to a glycoprotein through click-reaction. In certain embodiments, the agent-glycoprotein complex may be present intracellularly. In some embodiments, a cell may comprise at least one, at least two, at least three, at least four, at least 5, at least 6, at least 7, at least 8, at least 9, or at least 10, or more agent/moieties attached to a glycoprotein through click-reaction, optionally, wherein each agent/moiety is the same, or wherein each agent/moiety is different.

In some embodiments, the present invention provides a plurality of cells (e.g., a population of cells) comprising an agent/moiety attached to a glycoprotein through click-reaction. In certain embodiments, the agent-glycoprotein complex may be present intracellularly. In some embodiments, a plurality of cells (e.g., a population of cells) may comprise at least one, at least two, at least three, at least four, at least 5, at least 6, at least 7, at least 8, at least 9, or at least 10, or more agent/moieties attached to a glycoprotein through click-reaction, optionally, wherein each agent/moiety is the same, or wherein each agent/moiety is different.

In an exemplary embodiment, the click-coupled moieties are targeted to click-labeled stem cells, e.g., human embryonic stem cell. In another exemplary embodiment, the click-coupled moieties are targeted to click-labeled neural cells, e.g., human neural crest cells. In still another embodiment, the click coupled moieties are targeted to click-labeled fibroblast cells, e.g., human dermal fibroblast cells. In an exemplary embodiment, the click-coupled moieties are targeted to click-labeled undifferentiated cells, e.g., undifferentiated stem cells.

In one embodiment, the click-coupled moiety is a protein, a peptide, a nucleic acid, or a small molecule. In an exemplary embodiment, the click-coupled moiety is a protein or a peptide, or a polynucleotide encoding the protein or peptide. When the moiety refers to a protein or a peptide, it is contemplated that the polynucleotide encoding such protein or a peptide is also a click-coupled moiety. Non-limiting exemplary protein or peptide includes, but is not limited to, transcriptional factor, growth factor, cytokine, antibody, and/or gene editing molecules. The protein or peptide may be a fusion protein that comprises a reporter protein or peptide, e.g., GFP, to facilitate the screening and/or selection of cells that are targeted by the click-coupled moiety.

In some embodiments, the click-coupled moiety is a nucleic acid. The nucleic acid may be synthesized to incorporate a reactive group, such as an amine or thiol group, to be conjugated to a click reagent, e.g., DBCO by reacting with DBCO-NHS or DBCO-maleimide.

Using this approach, cells can be covalently coupled to a detectable label. For example, click-labeled cells can be contacted with a detectable label coupled to a second click reagent, which selectively reacts with the click reagent on the click-labeled cells. In embodiments where cells are covalently coupled to a detectable label in vivo, this can be accomplished by administering the detectable label coupled to the second click reagent to a subject. The detectable label can be a fluorescent label. Exemplary fluorescent labels include, but are not limited to, Alexa Fluor (e.g., Alexa Fluor 405, Alexa Fluor 488, Alexa Fluor 700, Alexa Fluor 750, etc.), GFP, FITC, CFSE, DyLight 488, phycoerythrin (PE), propidium iodide (PI), PerCP, Cy5, Cy5.5, Cy7, APC-eFluor 780, Draq-5, APC, amine aqua, pacific orange, pacific blue, DAPI, eFluor 450, eFluor 605, eFluor 625, and eFluor 650. In other embodiments, the detectable label can be a radiolabel. Exemplary radiolabels include, but are not limited to, ³H, ¹⁴C, ¹³N, ¹⁵O, ¹⁸F, ³²P, ³⁵S, ^{99m}Tc, ¹²³I, ¹²⁵I, and ⁶⁷Ga.

In other embodiments, cells can be covalently coupled to a moiety that modifies the genome or gene expression profile of the cells. For example, the click-coupled moiety is a transcription factor that modulates the expression of one or more genes in the cells. In some embodiments, the click-coupled moiety is oligonucleotides that can inhibit the expression of gene, e.g., siRNAs, shRNAs, or a ribozyme.

In certain embodiments, the click-coupled moiety comprises one or more components of a gene editing system. Exemplary gene editing systems include, but are not limited to meganucleases, zinc finger nucleases (ZFNs), transcription ctivator-like effector-based nucleases (TALEN), and the clustered regularly interspaced short palindromic repeats (CRISPR/Cas9) system. In some particular embodiments, the click-coupled moiety is CRISPR associated protein (Cas9), e.g., Cas9. The click-coupled moiety may also be a single guide RNA (sgRNA) used in a CRISPR/Cas9 gene editing system. Current methods to deliver proteins, e.g., Cas9, gRNAs, or gRNA/Cas9 complex to human stem cells and primary cells, via passive internalization, are usually inefficient. Advantageously, the approach according to the invention, e.g., targeting molecules, such as Cas9, gRNA, or gRNA/Cas9 complex using click chemistry, and inducing endocytosis of the molecule-glycoprotein complex, improves the intracellular delivery efficiency.

Accordingly, in certain embodiments, the present invention provides methods of gene editing or genome editing. The methods comprise contacting a cell comprising surface glycoprotein coupled with a click reagent with a counterpart click reagent that is, in turn, attached to a moiety, thereby conjugating the moiety to the cell. The click reagent coupled moiety comprising a component of a gene editing system. In some embodiments, the gene editing system is selected from the group consisting of meganucleases, zinc finger nucleases (ZFNs), transcription ctivator-like effector-based nucleases (TALEN), and the clustered regularly interspaced short palindromic repeats (CRISPR/Cas9) system. Without wishing to be bound by any theory, it is hypothesized that gene editing moiety coupled to a click reagent may be covalently linked to a counterpart click reagent that is coupled to a cell surface glycoprotein. The gene editing moiety-cell surface glycoprotein complex is engulfed into the cell through endocytosis. The gene editing moiety may then dissociate from the complex and exert its effect on one or more target genes or one or more genome loci. In certain embodiments, the gene editing moiety is coupled to a click reagent, e.g., DBCO, through a linker. The linker may be a disulfide bond, hydrozone, bond, or enzyme cleavable bond. The linker can be cleaved in endosome, facilitating the gene-editing moiety to be released from endosome. In some particular embodiments, the gene editing system is CRISPR/Cas9 system. The gene editing moiety is Cas9 and/or sgRNA that is specific to one or more genes or one or more genome loci to be edited. In some other particular embodiments, the gene editing moiety is Cas9. In some embodiments that CRISPR/Cas9 system is targeted to cell, the sgRNA may be introduced into a cell independently. The sgRNA may be targeted to a cell intracellularly via an independent click reaction, e.g., sgRNA coupled to a click reagent. The sgRNA may also be targeted to a cell using any method known in the art, e.g., transfection. Any well known techniques in the art may be used to transfect a sgRNA to a cell.

Accordingly, cells, e.g., stem cells, may be manipulated using click chemistry. Such a manipulation may be transient or stable. For example, in some embodiments, the agent targeted to a cell may be a transcriptional factor, which alters the gene expression profile of the cell transiently. In certain embodiments, the manipulation may be stable. For example, the agent targeted to cell may be gene editing molecules, e.g., Cas9. The gene editing molecules may edit one or more genes or one or more loci on the genome, which can be passaged to future generations of the cell.

In some embodiments, the present invention provides a method to modulate, e.g., enhance or suppress, gene expression of a cell. The method comprises contacting a cell coupled to a click reagent with a transcriptional regulator/factor coupled to a counterpart click reagent. As used herein, transcriptional regulator includes any agent that can modulate the expression of one or more genes directly or indirectly. The transcriptional regulator may be a small molecule, a peptide, a protein, or a nucleic acid.

In some embodiments, the transcriptional regulator is a transcriptional factor that directly binds to transcriptional regulatory area of one or more genes to modulate the expression of the one or more genes. In some other embodiments, the transcriptional regulator is an agent, e.g., a protein, that modulates the signal pathway that leads to the modulation of the expression of one or more genes. For example, the transcriptional regulator may be a protein kinase that phosphorylates another protein, which, in turn, leads to the modulation of expression.

Still in certain embodiments, the transcription regulator may exert its effect through a component of a signal cascade that modulates the gene expression profile. For example, the transcription regulator may be an antibody that blocks the interaction between two or more proteins that are involved in a signal cascade.

The transcriptional regulator/factor may be a protein or a peptide or a polynucleotide encoding such a protein or peptide. The transcriptional regulator/factor may also be a nucleic acid, e.g., siRNA, shRNA, iRNA, or miRNA, that modulates the expression of one or more genes.

In particular embodiments, the present invention provides a method of inducing cell differentiation. The method comprises contacting a cell, e.g., a stem cell, coupled to a click reagent with a differentiation inducing agent coupled to a counterpart click reagent. As used herein, a “differentiation inducing agent” is an agent that induces a pluripotent cell, e.g., stem cell, to differentiate into a specialized cell, e.g., a neuron, an epithelial cell, a fibroblast, or an immune cell. Differentiation includes, but is not limited to, direct differentiation, transdifferentiation, and cell reprogramming. In certain embodiments, the differentiation inducing agent is a transcriptional regulator/factor that modulates the expression of one or more genes. The change in gene expression profile in turn induces the differentiation of the cell. Exemplary transcriptional regulator/factor is described in Oh et al., Directed differentiation of pluripotent stem cells by transcription factors, 42(3):200-209 (2019), incorporated herein by reference.

In certain embodiments, the differentiation inducing agent may be a gene editing molecule, e.g., Cas9. The gene editing molecule may edit one or more genes or one or more loci in the genome of the cell. The gene editing may thus alter the gene expression profile of gene and induce differentiation of the cell.

In some embodiments, the differentiation inducing agent may be a protein, e.g., a growth factor that binds to a receptor and initiates the signal transduction cascade that leads to the differentiation of a cell. For example, the growth factor may include basic fibroblast growth factor, transforming growth factor β1 (TGF-β1), activin-A, bone morphogenic protein 4 (BMP-4), hepatocyte growth factor (HGF), epidermal growth factor (EGF), or β nerve growth factor (βNGF). In such a situation, the growth factor may remain on the surface of the cell and bind to its receptor. Without wishing to be bound by any theory, it is hypothesized the growth factor, by virtue of covalently attached to the glycoprotein coupled to a click agent, may provide sustained and prolonged activation/inactivation of certain receptor, thereby enhancing the differentiation of a cell. In still some other embodiments, the differentiation inducing agent may be a component of the signal cascade that is involved in the differentiation of a cell. For example, the differentiation inducing agent may be a Notch signaling pathway component, such as Notch ligands, Delta-like ligands.

In certain embodiments, the differentiation inducing agent may exert its effect through a component of a signal cascade that leads to the differentiation of a cell. For example, the differentiation inducing agent may be an antibody that blocks interaction between two or more proteins to induce the differentiation of the cell.

In certain embodiments, the differentiation inducing agent may be a small molecule. For example, the differentiation inducing agent may be retinoic acid that facilitates the differentiation to neuron.

In certain embodiments, the cells are manipulated in vitro or ex vivo. The targeting of cells using click chemistry pairs may be combined with other treatment or manipulation of the cells. For example, cells may be screened, selected, expanded and/or differentiated after a click-couple moiety is targeted to the cells.

Advantageously, the click reagent coupled moiety can be specifically targeted to a cell comprising surface glycoprotein coupled with a counterpart click reagent, thereby increasing the specificity of the targeting of the moiety. This is particularly useful for targeting a cell that is labeled with a click reagent in vivo. For example, a cell may be metabolically labeled with a click reagent in vitro and administered to a subject. A moiety coupled to a counterpart click reagent may be administered to a subject separately. The metabolically labeled cell can specifically be linked to the moiety coupled to a click reagent through click chemistry in vivo. The administration of the click reagent-coupled moiety may be prior to, concurrently with, or after the administration of the cell labeled with a counterpart click reagent.

For in vivo targeting an agent to a cell, a moiety coupled to a click reagent can be administered to a subject, e.g., a subject comprising click-coupled cells, by any suitable method. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by injection, e.g., subcutaneous injection or intratumoral injection, or by intravenous infusion.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

In some embodiments, the administration is by subcutaneous injection. In some embodiments, the administration is by intravenous injection.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject. In one embodiment, the amount is a therapeutically effective amount.

The term “therapeutically effective amount”, as used herein, means that amount of a compound, material, or composition comprising a compound described herein which is effective for producing some desired therapeutic effect in at least a sub-population of cells in a subject at a reasonable benefit/risk ratio applicable to any medical treatment. Thus, “therapeutically effective amount” means that amount which, when administered to a subject for treating a disease, is sufficient to effect such treatment for the disease.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

In one embodiment, the dosage is a weight-based dose. In exemplary embodiments, the weight-based dose is 0.001-100 mg/kg. For example, in some embodiments, the dosage is 0.001-0.1 mg/kg. In other embodiments, the dosage is 0.01-1 mg/kg. In other embodiments, the dosage is 0.1-10 mg/kg. In other embodiments, the dosage is 1-100 mg/kg. In other embodiments, the dosage is about 1 mg/kg, 2 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, or 100 mg/kg.

The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

### V. PHARMACEUTICAL COMPOSITIONS

For administration to a subject, the polymers, nanoparticles, devices, scaffolds, hydrogels, agents coupled to click chemistry reagents, and cells described herein can be provided as pharmaceutically acceptable (e.g., sterile) compositions. Accordingly, in one aspect, the invention provides a pharmaceutical composition comprising a polymer or nanoparticle comprising a click reagent.

These pharmaceutically acceptable compositions can be formulated together with one or more pharmaceutically acceptable carriers (additives) and/or diluents. As described in detail below, the pharmaceutical compositions of the present disclosure can be specifically formulated for administration in solid or liquid form, including those adapted for the following: (1) parenteral administration, for example, by subcutaneous, intramuscular, intravenous (e.g., bolus or infusion) or epidural injection as, for example, a sterile solution or suspension, or sustained-release formulation; (2) oral administration, for example, drenches (aqueous or non-aqueous solutions or suspensions), lozenges, dragees, capsules, pills, tablets (e.g., those targeted for buccal, sublingual, and/or systemic absorption), boluses, powders, granules, pastes for application to the tongue; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by FDA Office of Biological Standards.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable carrier” or the like are used interchangeably herein.

The pharmaceutical compositions of the invention comprising a click reagent can be delivered to an in vivo locus in a subject. Exemplary in vivo loci include, but are not limited to site of a wound, trauma or disease. The composition can be delivered to the in vivo locus by, for example, implanting the compositions into a subject. The composition can optionally include one or more additives. Additives can include, but are not limited to, resolving (biodegradable) polymers, mannitol, starch sugar, inosite, sorbitol, glucose, lactose, saccharose, sodium chloride, calcium chloride, amino acids, magnesium chloride, citric acid, acetic acid, hydroxyl-butanedioic acid, phosphoric acid, glucuronic acid, gluconic acid, poly-sorbitol, sodium acetate, sodium citrate, sodium phosphate, zinc stearate, aluminium stearate, magnesium stearate, sodium carbonate, sodium bicarbonate, sodium hydroxide, polyvinylpyrolidones, polyethylene glycols, carboxymethyl celluloses, methyl celluloses, starch or their mixtures.

### VI. KITS

Any of the compositions described herein may be comprised in a kit. In a non-limiting example, the kit comprises a click functionalized polysaccharide polymer which is a product of radical-catalyzed polymerization. In certain embodiments, the kit includes nanoparticles for labeling cells with a click reagent comprising the click functionalized polysaccharide polymer. In certain embodiments, the kit comprises a click functionalized polysaccharide polymer which is a product of radical-catalyzed polymerization and a second click chemistry reagent coupled to an agent targeted to the cells, wherein the second click chemistry reagent can selectively react with the click reagent present in the functionalized polysaccharide polymer. In some embodiments, the kit includes nanoparticles for labeling cells with a click reagent comprising the click functionalized polysaccharide polymer and a second click chemistry reagent coupled to an agent targeted to the immune cell, wherein the second click chemistry reagent can selectively react with the click reagent present in the nanoparticle.

The kit may further include reagents or instructions for in vivo labeling a cell in a subject and/or in vitro labeling a cell with a click chemistry reagent described elsewhere herein. It may also include one or more buffers. Other kits of the invention may include components for assays to detect the labeling of the cell. In certain embodiments, the kits of the invention comprise the reagents for detecting a detectable label that is targeted to a cell.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the compositions of the invention, e.g., the click functionalized polysaccharide polymer, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The present invention is further illustrated by the following examples, which should not be construed as limiting. The entire contents of all of the references cited throughout this application are hereby expressly incorporated herein by reference.

### EXAMPLES

### Example 1: Poly(Azido-Sugar) for Metabolic Labeling of Cells

It was first tested whether poly(azido-sugar) can label a variety of cells with azido groups. The methods to prepare tetraacetyl-N-azidoacetylmannosamine (Ac₄ManAz) and poly (azido-sugar) has been described in International Patent Application PCT/US2019/051621, incorporated by reference. Briefly, Ac₄ManAz was synthesized following the reported procedure (Wang, H. et al. Selective in vivo metabolic cell-labeling-mediated cancer targeting. Nature Chemical Biology 13, 415-424 (2017)).

To prepare poly(azido-sugar), the C1 site of Ac₄ManAz was functionalized with an acrylate bond, followed by reversible addition-fragmentation chain transfer (RAFT) polymerization using poly(ethylene glycol) methyl ether 2-(dodecylthiocarbonothioylthio)-2-methylpropionate as the RAFT agent and azobisisobutyronitrile as the initiator to yield poly(azido-sugar)ₙ (n=400 (G400)). Briefly, Ac₄ManAz (1 mmol) was dissolved in methanol/tetrahydrofuran (1/2, v/v), followed by the addition of ammonium carbonate (1.2 mmol). The reaction mixture was stirred at room temperature for 24 hours. After removal of the solvent under reduced pressure, the crude product was purified by silica gel column chromatography to yield Ac₃ManAzOH. Ac₃ManAzOH (1.0 mmol) was then dissolved in dry dichloromethane, followed by the addition of acryloyl chloride (3.0 mmol) and triethylamine (1.0 mmol). The reaction mixture was stirred at room temperature for 24 hours. After removal of the solvent and residual acryloyl chloride, the crude product was redissolved in dichloromethane, washed with deionized water for three times, and dried to yield Ac₃ManAzAL. Ac₃ManAzAL (1.0 mmol), azobisisobutyronitrile (AIBN, 0.008 or 0.0005 mmol), and poly(ethylene glycol) methyl ether 2-(dodecylthiocarbonothioylthio)-2-methylpropionate (PEG DDMAT, 0.04 or 0.0025 mmol) were dissolved in anhydrous DMF, followed by three freeze-thaw cycles and stirring at 65° C. for 48 hours. Poly(azido-sugar) (G400) was obtained via precipitation in cold diethyl ether, washed with diethyl ether for three times, and dried under reduced pressure. Fluorescently labeled G25 and G400 were prepared via conjugation of DBCO-dyes to G25 and G400, respectively (1 mg).

G400 NP were then prepared via nanoprecipitation of G400. Briefly, G400 polymer was dissolved in DMF at a concentration of 40 mg/mL, and dropwise added to ultrapure water (20-fold volume) upon vigorous stirring. After stirring for 4 hours, G400 NP solution was dialyzed against deionized water for 48 hours, sterilized, and then stored at 4° C. for use. Dye-labeled G400 NP were prepared similarly using dye-labeled G400, respectively.

To metabolically label human embryonic stem cells (hESCs), hESCs were incubated with G400 NP for three days and staining with DBCO-Cy3 for 20 minutes at 37° C. hESCs showed significantly enhanced Cy3 signal compared to control cells without G400 NP pretreatment (FIGS. 2A-2C).

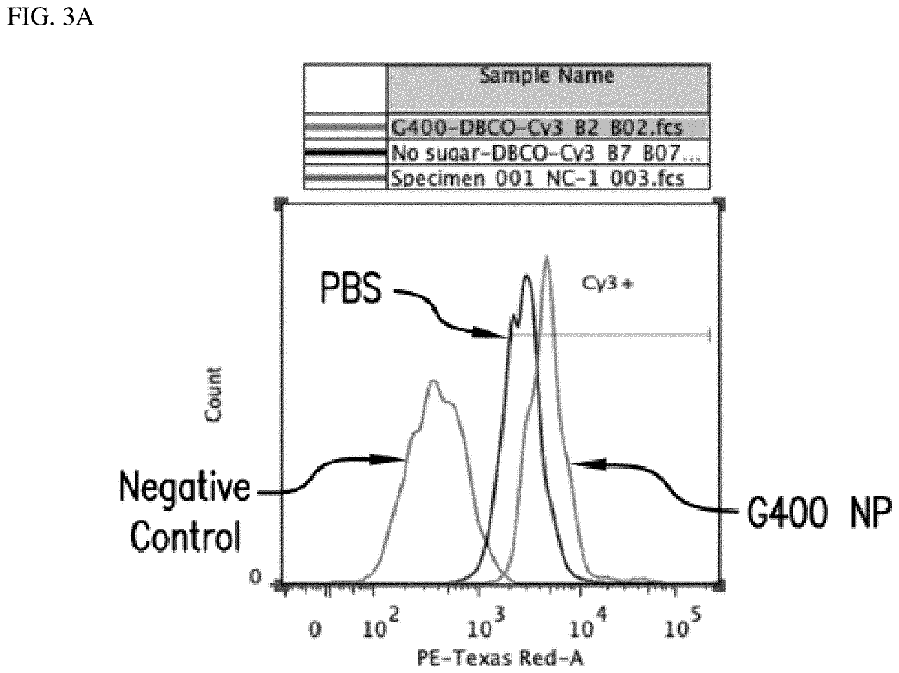

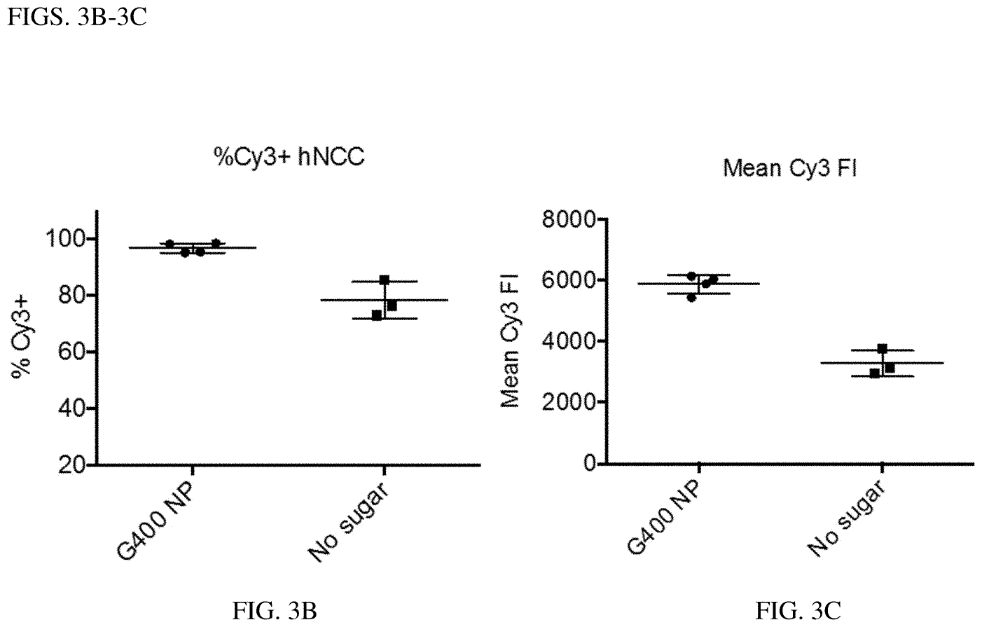

To metabolically label human neural crest cells (hNCCs), hNCCs were incubated with G400 NP for three days and staining with DBCO-Cy3 for 25 minutes at 37° C. hNCCs showed significantly enhanced Cy3 signal compared to control cells without G400 NP pretreatment (FIGS. 3A-3C).

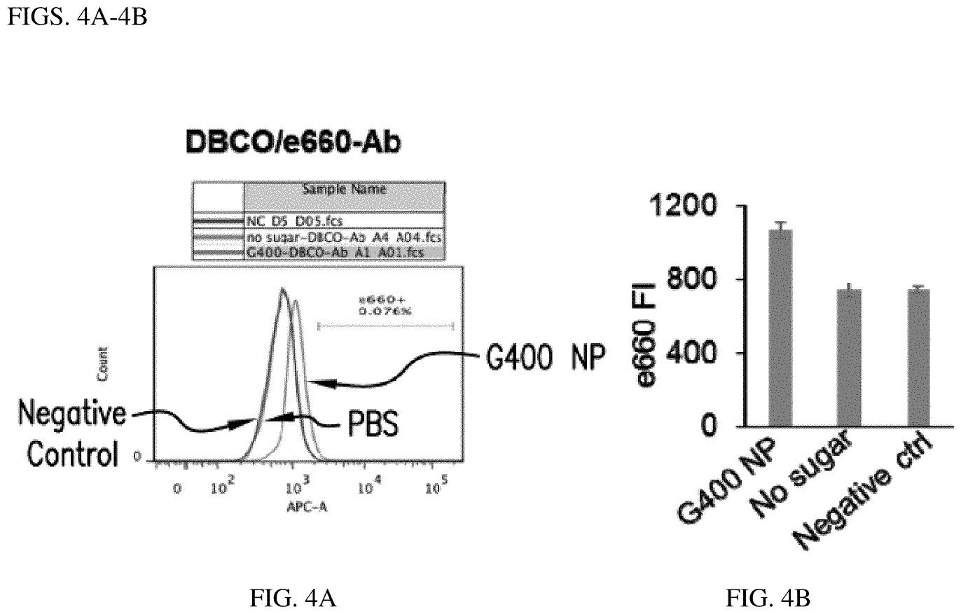

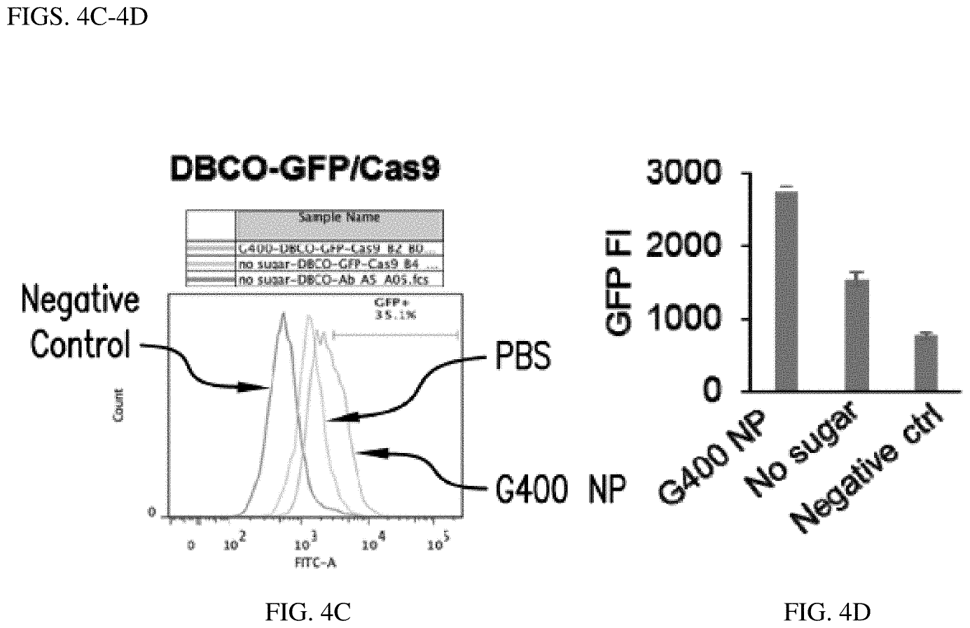

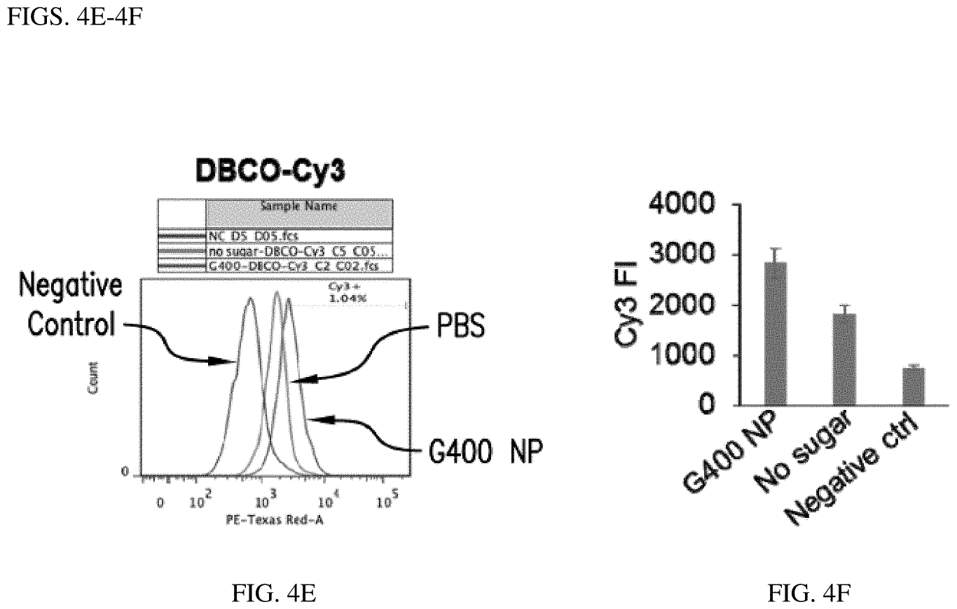

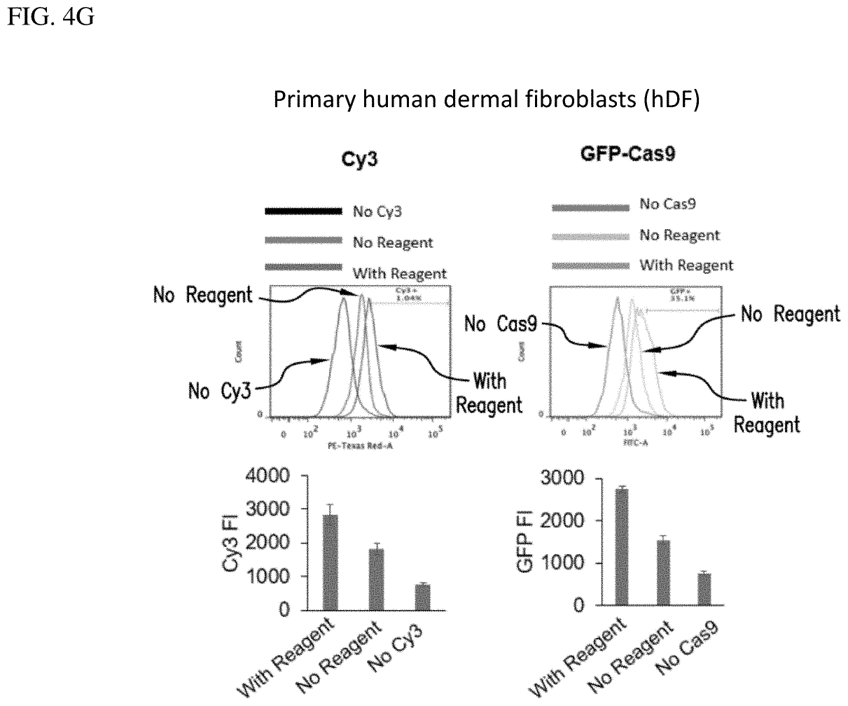

To metabolically label human dermal fibroblasts (hDFs), hDFs incubated with G400 NP for four days and staining with DBCO-Cy3 for 25 minutes at 37° C. hDFs showed significantly enhanced Cy3 signal compared to control cells without G400 NP pretreatment (FIG. 4F). In comparison, a DBCO- and efluor660-conjugated rat IgG2a isotype control antibody (DBCO/efluor660-antibody) also showed significantly enhanced e660 signal compared to control cells without G400 NP pretreatment (FIG. 4B).

For flow cytometry analysis of azido-labeled cells, cells were cultured and labeled as described above. After washing with PBS, cells were incubated with DBCO-Cy3, DBCO/efluor660-antibody, or DBCO-GFP/Cas9 for 20-30 minutes on ice. Cells were then collected via a cell scraper, re-suspended in FACS buffer, and analyzed by flow cytometry. FIGS. 2A, 3A, 4A, 4C, and 4E are graphs from flow cytometry analysis that show cells can be metabolically labeled with G400 NP and specifically targeted by moieties coupled to DBCO.

### Example 2: Delivery of GFP/Cas9 Via Click Chemistry

It was next investigated whether human dermal fibroblasts (hDFs) labeled with azide can be targeted with Cas9 coupled to DBCO.

hDFs were cultured and labeled as described in Example 1. To target GFP/Cas9 fusion protein to the azide-labeled hDFs, hDFs were incubated with DBCO-GFP/Cas9 at 37° C. for 25 minutes. hDFs were then subject to flow cytometry or detection of GFP/Cas9 as described in Example 1. hDFs showed significantly enhanced GFP signal compared to control cells without G400 NP pretreatment (FIG. 4D). As shown in FIG. 4C and FIG. 4G, hDF can be metabolically labeled with G400 NP and specifically targeted by GFP/Cas9 coupled to DBCO.

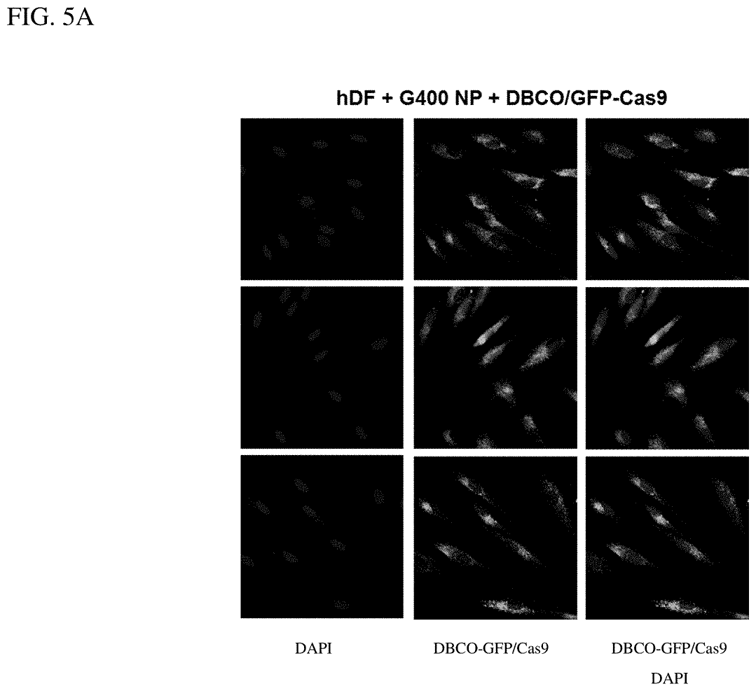

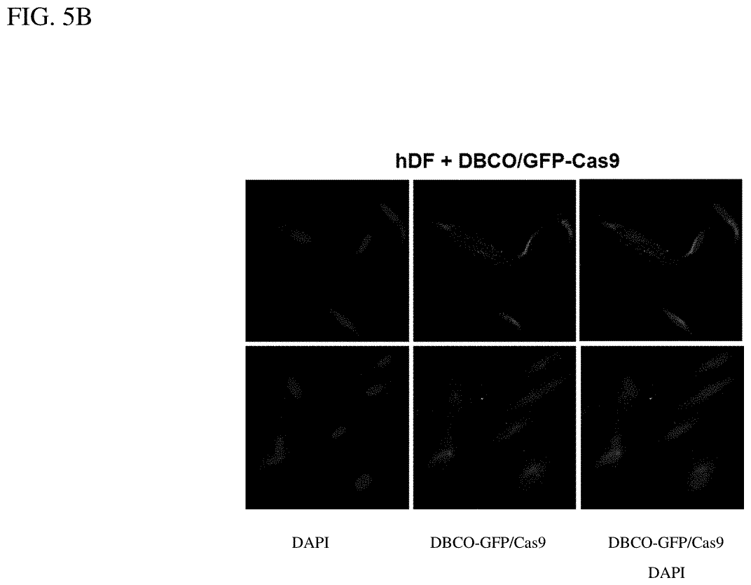

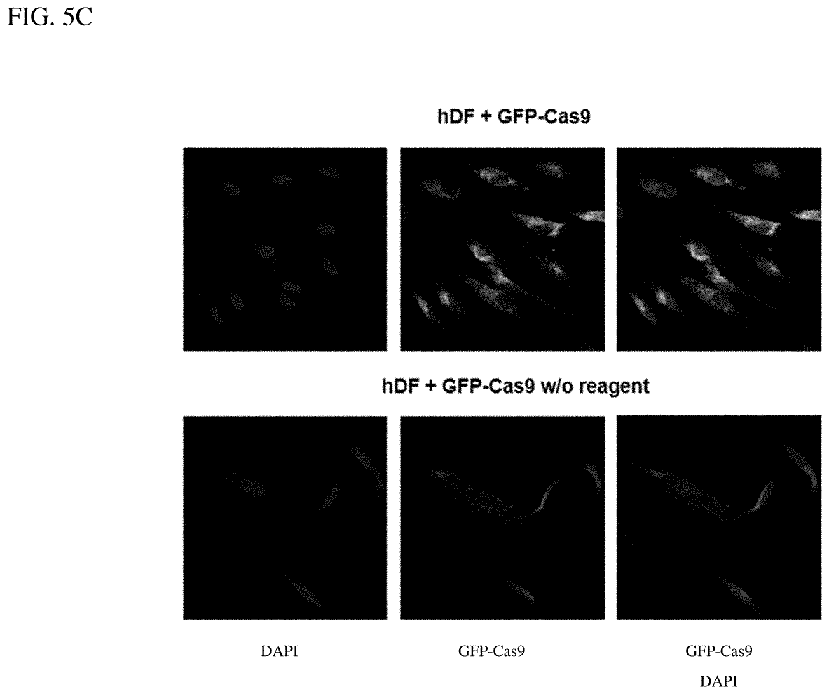

The specific targeting of DBCO-GFP/Cas9 to hDFs labeled with azide was further confirmed by confocal images. As shown in FIG. 5A and FIG. 5C, GFP signal was detected in almost all the hDFs that were labeled with azide. By contrast, as shown in FIG. 5B and FIG. 5C, GFP/Cas9 rarely entered cells without sugar labeling.

### Example 3: Delivery of GFP Modified with Different Linkers to the Cytoplasm of Primary Adult Human Dermal Fibroblasts

It was next investigated whether delivery of recombinant green fluorescent protein (EGFP) can be targeted to the cytoplasm of primary adult human dermal fibroblasts using EGFP modified with either a DBCO-NHS linker (DBCO-GFP) or a reducible linker DBCO-disulfide-NHS (DBCO-SS-GFP).

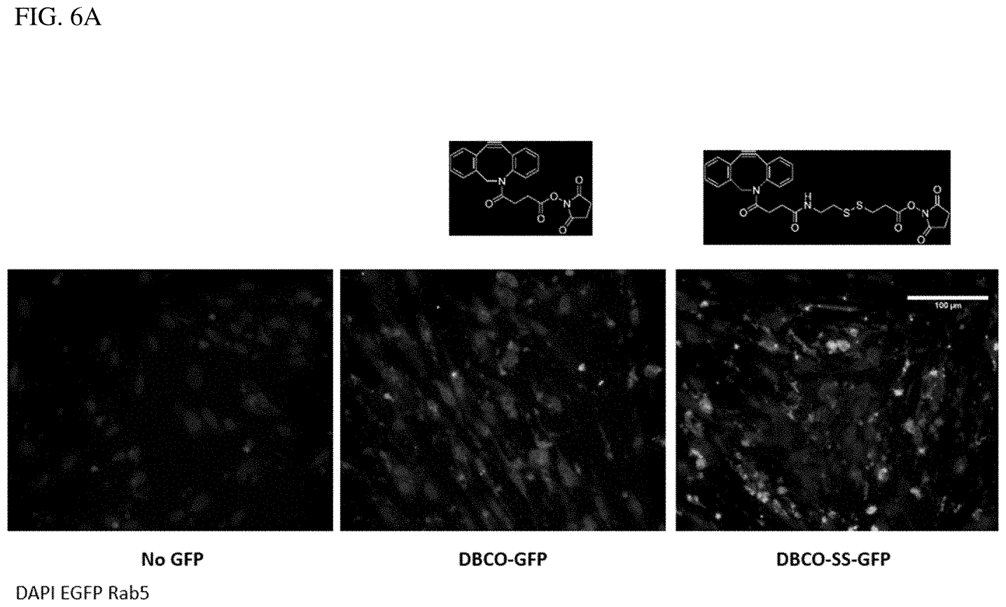

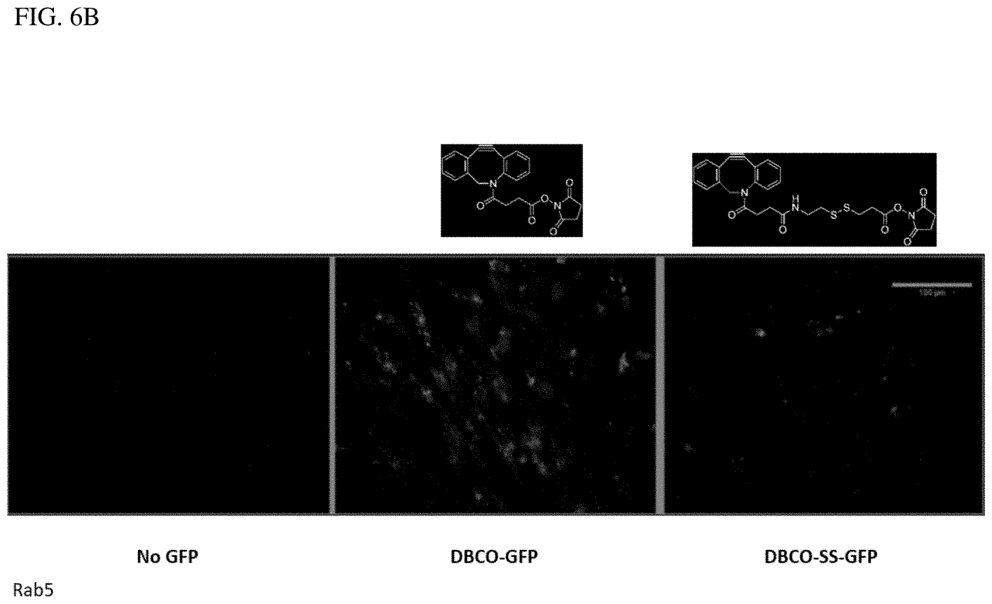

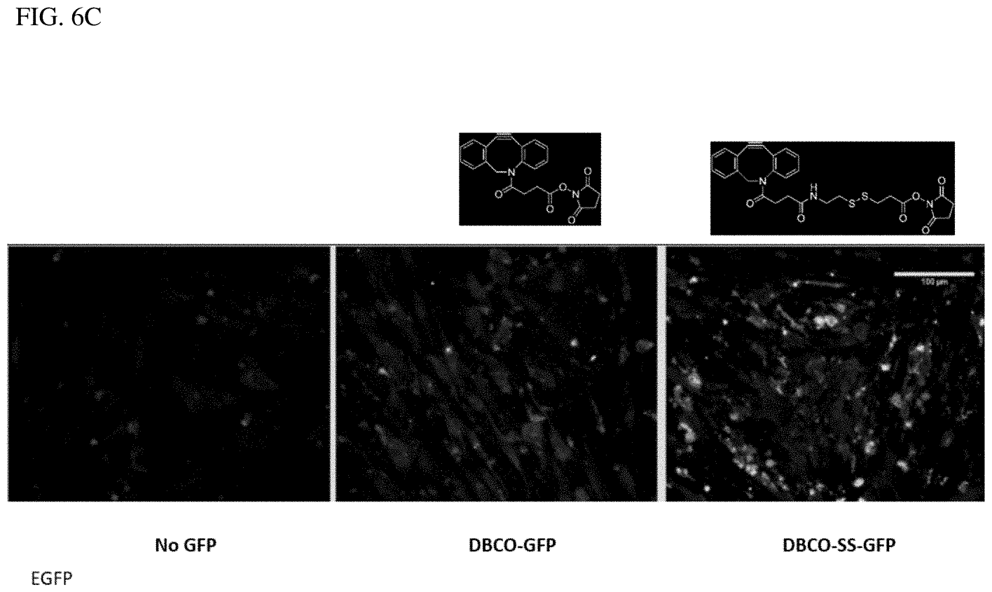

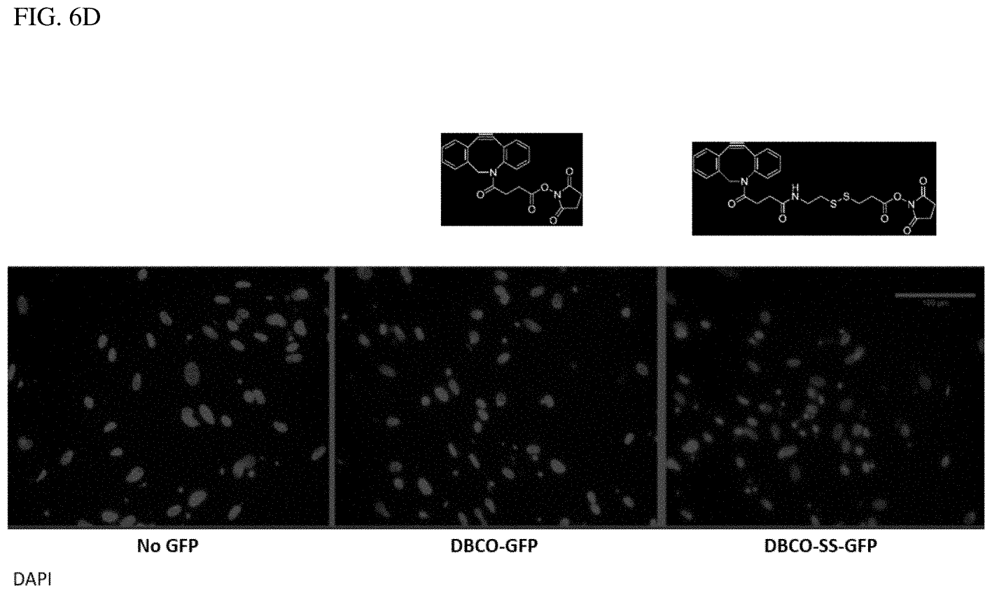

As shown in FIGS. 6A-6D, cytoplasmic delivery of recombinant green fluorescent protein (EGFP) to primary adult human dermal fibroblasts was detected. Endosomes were stained for the marker RabS and showed no colocalization with EGP signal, indicating cytoplasmic delivery.

### Example 4: Delivery of GFP Modified with Different Linkers to Stem Cell-Derived Adipocytes

It was next investigated whether delivery of recombinant green fluorescent protein (EGFP) can be targeted to stem cell-derived adipocytes using EGFP modified with either a DBCO-NHS linker (DBCO-GFP) or a reducible linker DBCO-disulfide-NHS (DBCO-SS-GFP).

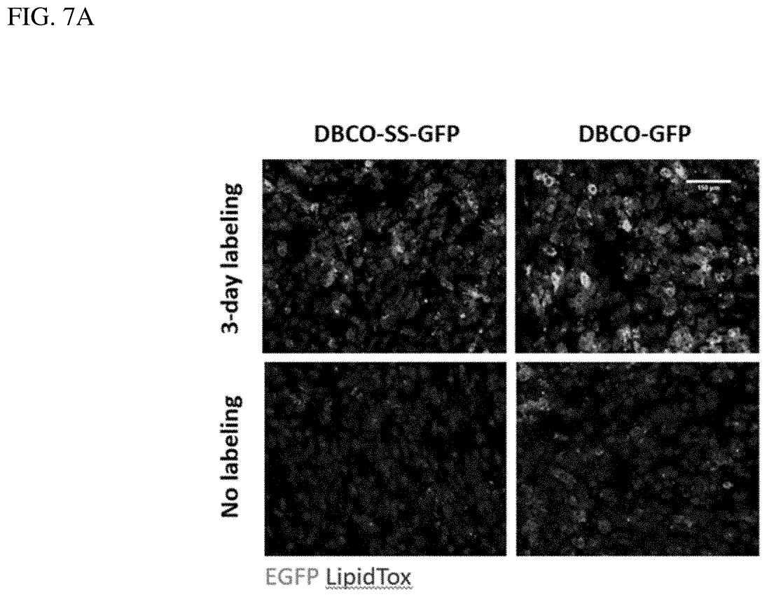

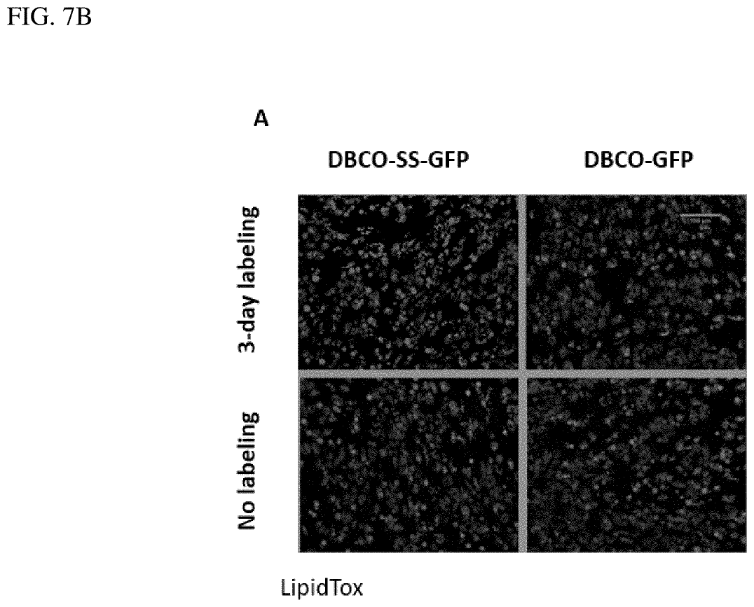

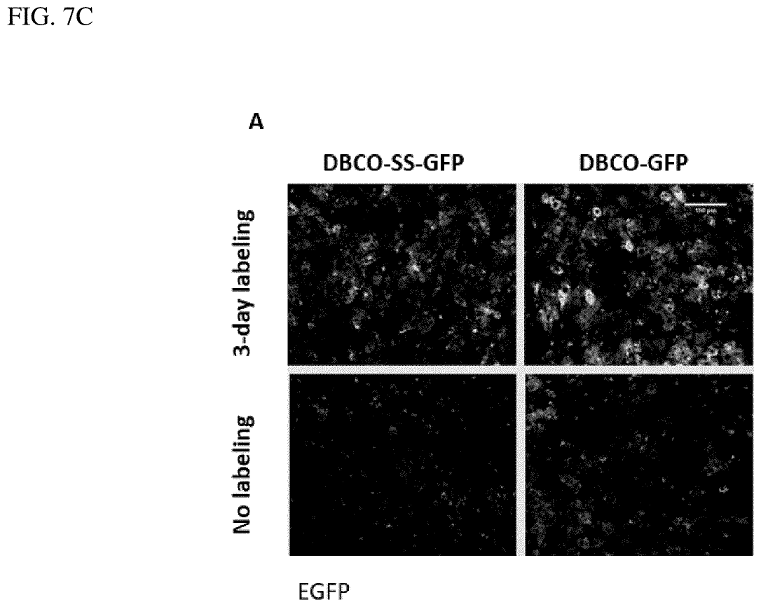

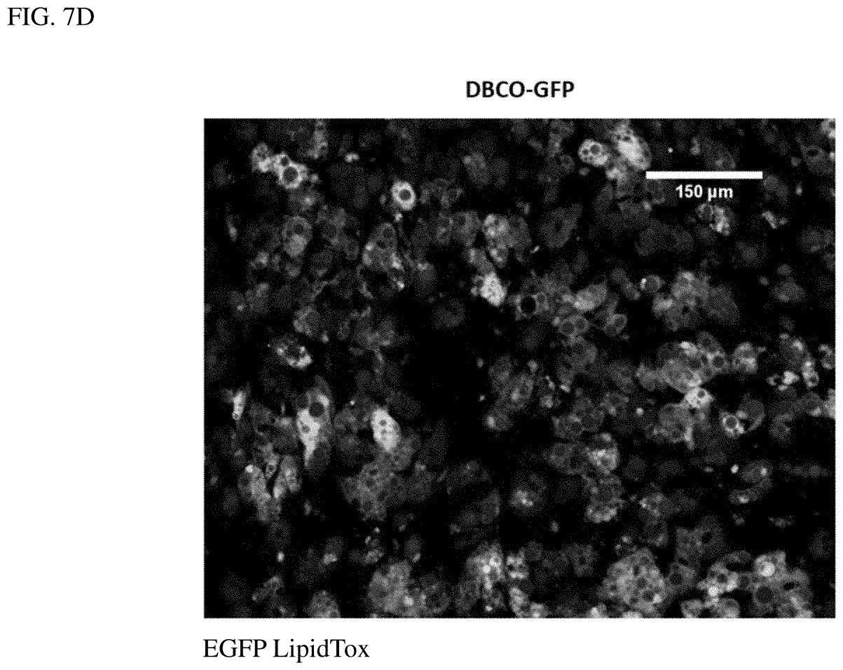

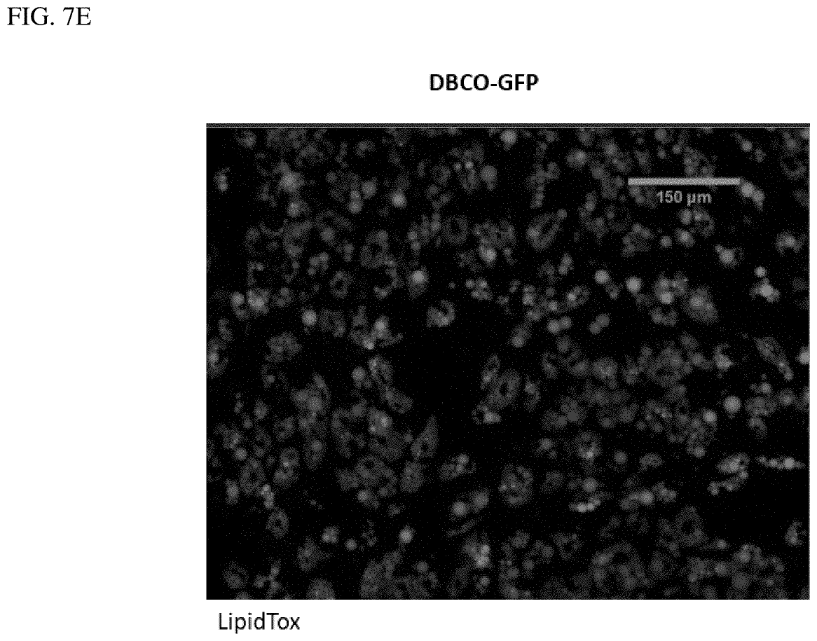

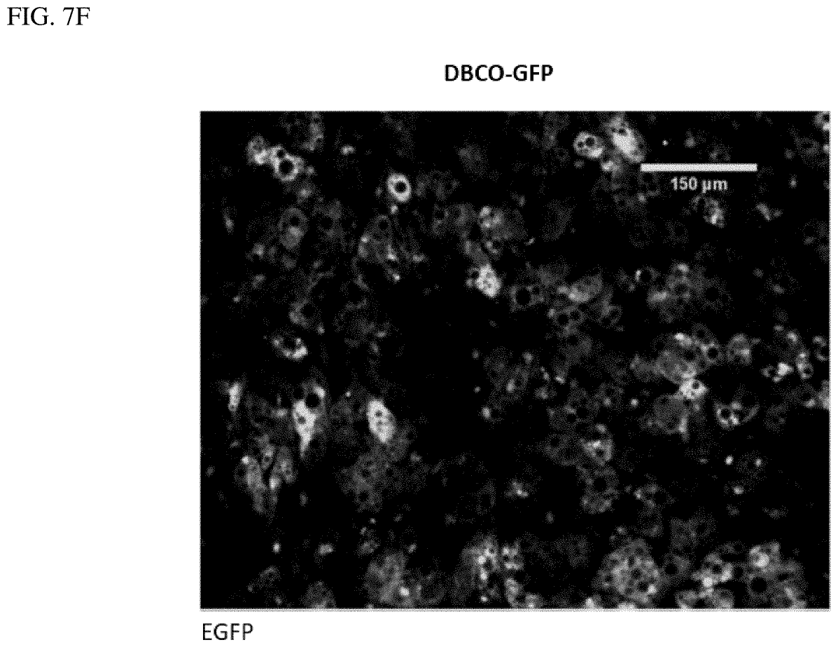

As shown in FIGS. 7A-7F, the cytoplasmic delivery of recombinant green fluorescent protein (EGFP) to stem cell-derived adipocytes was detected. Lipid droplets were stained with the neutral lipid stain, LipidTox, and showed no colocalization with EGP signal, indicating exclusion from intracellular vesicles, indicating cytoplasmic delivery.

### Example 5: Dibenzocyclooctyne-N-Hydroxysuccinimidyl Ester (DBCO-NHS) for Labeling Cas9-EGFP Proteins

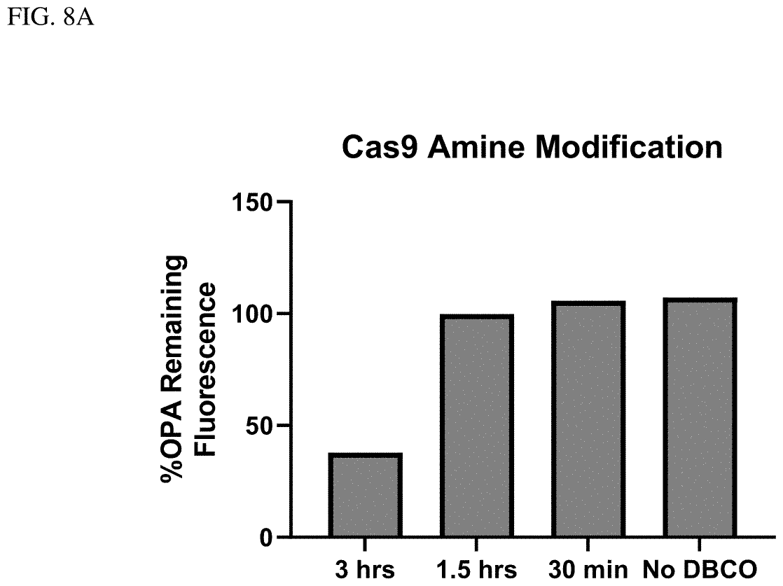

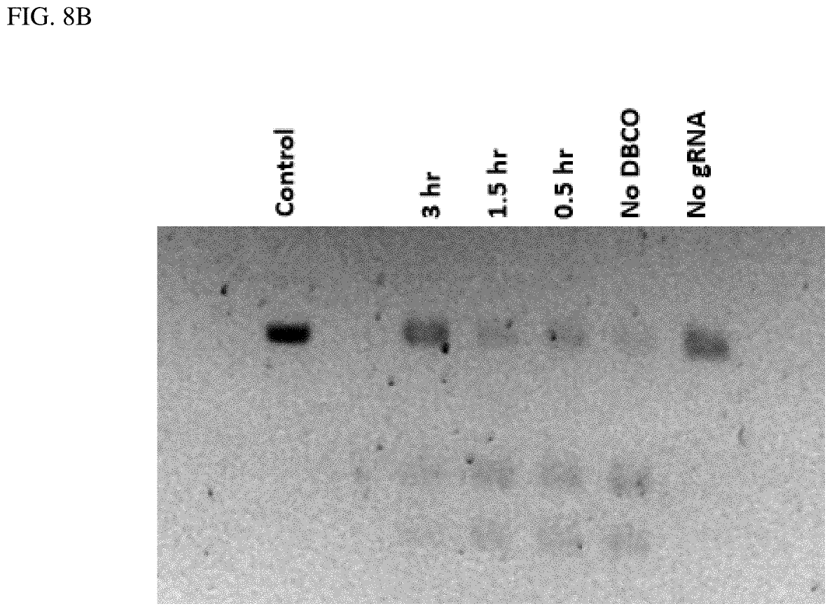

Recombinant Cas9-EGFP protein (Sigma Aldrich) was incubated at 3 μM in PBS and reacted with 30 μM Dibenzocyclooctyne-N-hydroxysuccinimidyl ester (DBCO-NHS) at room temperature for the times denoted in FIG. 8A. The reactions were purified by dilution and filtration of side-products through a 50 kDa amplicon centrifugal filter three-times. The modified Cas9-EGFP proteins were reacted with OPA-Fluoraldehyde to determine the number of reactive free amines to determine the degree of modification. The modified proteins were also characterized for the retention of their nuclease activity by incubating 30 nM of each protein with 30 nM of a sgRNA designed to target a GFP-encoding DNA sequence and 3 nM of a PCR product from a GFP-encoding plasmid. The incubations were run a 1.0 wt % agarose gel, and DNA fragments were visualized with ethidium bromide staining and ultraviolet illumination (FIG. 8B).

### Example 6: Dibenzocyclooctyne-N-Hydroxysuccinimidyl Ester (DBCO-NHS) for Labeling Oct4 Protein

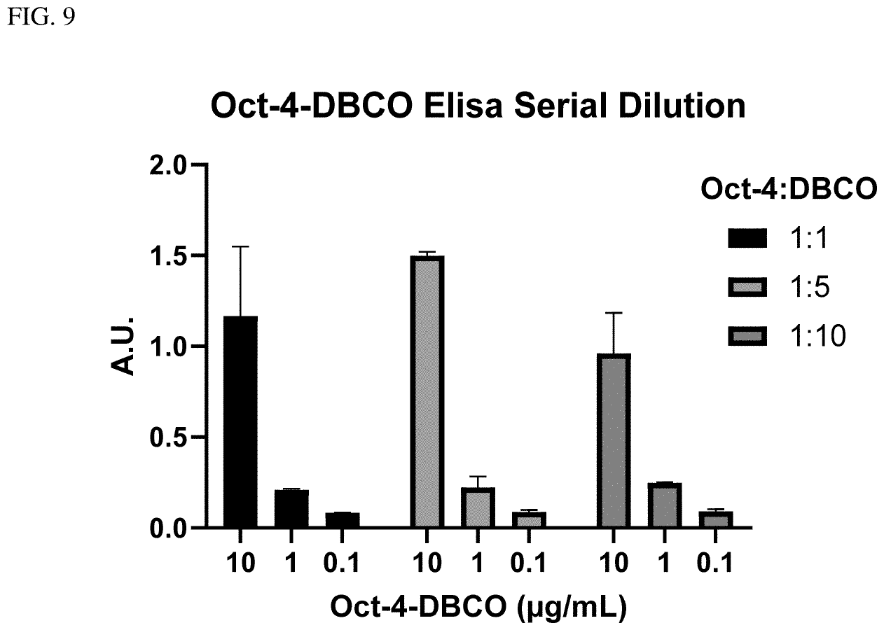

Recombinant Oct4 protein (Abcam) was incubated at 3 μM in PBS and reacted with 1, 5, and 10-fold equivalent molar ratios of Dibenzocyclooctyne-N-hydroxysuccinimidyl ester (DBCO-NHS) at 4° C. for the 24 hrs. The reactions were purified by dilution and filtration of side-products through a 50 kDa amplicon centrifugal filter three-times. Retention of DNA-binding capacity after modification was characterized with an oligonucleotide-based enzyme-linked immunosorbent assay (Ray Biotech) in which the oligonucleotide target is based on the genomic binding sequence for the Oct4 transcription factor (FIG. 9).

### Example 7: Dibenzocyclooctyne-N-Hydroxysuccinimidyl Ester (DBCO-NHS) for Labeling Cas9-EGFP Protein Complexed with sgRNA Targeting the ROSA26 Locus

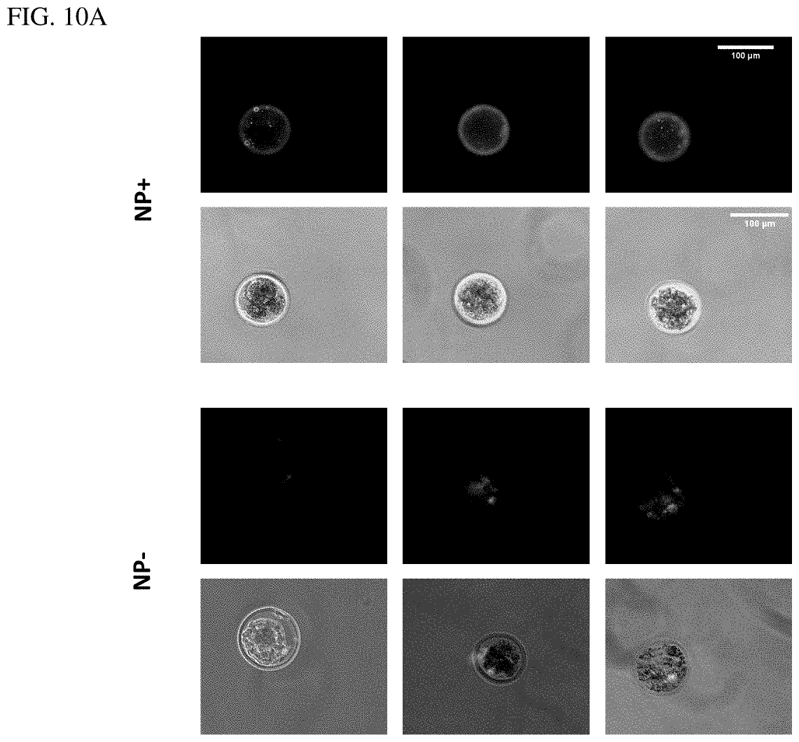

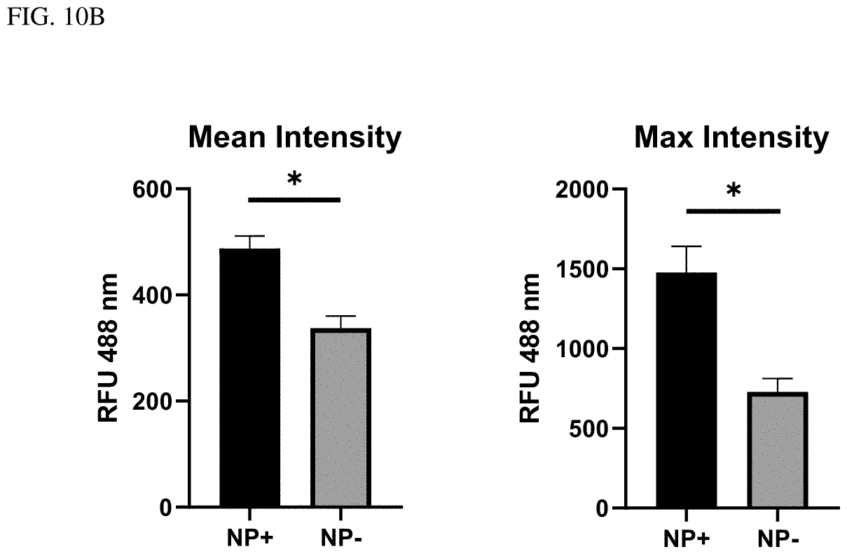

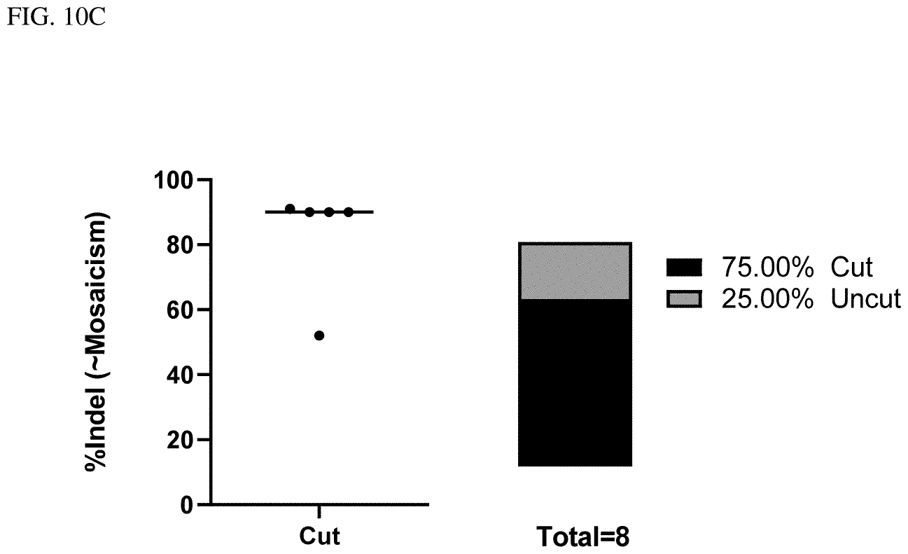

In vitro-cultured, live, mouse embryos were incubated with GP400 NP at 20 μM for 48 hours at 37° C. Recombinant Cas9-EGFP protein (Sigma Aldrich) or an AlexaFluor 488-labeled IgG antibody was incubated at 3 μM in PBS and reacted with 30 μM Dibenzocyclooctyne-N-hydroxysuccinimidyl ester (DBCO-NHS) at room temperature for 1.5 hours. The reactions were purified by dilution and filtration of side-products through a 50 kDa amplicon centrifugal filter three-times. Before culture, Cas9 was complexed with sgRNA targeting the ROSA26 locus. For both proteins, they were incubated with NP-labeled and control embyros for 3 hours. Embryos cultured with antibodies were fixed and imaged on a Nikon TE epifluorescence inverted microscope with FITC filter cube (FIG. 10A). Images were analyzed in Fiji, and mean and max fluorescence quantified (FIG. 10B). Embryos cultured with Cas9-EGFP/sgRNA, the embryo DNA was collected and the ROSA26 locus sequenced. Embryos containing errors we identified in alignment against a control sequence were used as evidence as indel presence due to Cas9 cutting, and % of read indel as indication of embryo mosaicism with respect to genome cutting (FIG. 10C).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A method of delivering an agent to a cell intracellularly, comprising contacting the cell coupled to a first click reagent with the agent coupled to a second click reagent, wherein the second click reagent selectively reacts with the first click reagent coupled to the cell, thereby delivering the agent to the cell intracellularly.

2. The method of claim 1, wherein the agent retains its structural integrity, function and/or activity after delivery to the cell intracellularly.

3. The method of claim 1, wherein the cell is contacted with the agent coupled to a second click reagent a plurality of times.

4. The method of claim 1, further comprising contacting the cell with an additional agent coupled to the second click reagent, optionally, wherein the additional agent coupled with the second click reagent contacts the cell prior to, concurrently with, or subsequent to contacting the cell with the agent coupled with the second click reagent.

5. The method of claim 1, wherein the cell is further coupled to a third click reagent, further comprising contacting the cell with an agent coupled to a fourth click reagent, wherein the fourth click reagent selectively reacts with the third click reagent, optionally, wherein the agent coupled to the fourth click reagent contacts the cell prior to, concurrently with, or subsequent to contacting the cell with the agent coupled with the second click reagent.

6. The method of claim 1, comprising selectively delivering an agent to a cell in a plurality of cells intracellularly, wherein the cell is coupled with a first click reagent, comprising contacting the plurality of cells with the agent coupled to a second click reagent, wherein the second click reagent selectively reacts with the first click reagent coupled to the cell, thereby delivering the agent selectively to the cell intracellularly.

7. (canceled)

8. The method of claim 1, wherein the at least one agent induces the differentiation of the cell coupled with the first click reagent.

9. (canceled)

10. The method of claim 1, wherein the click reagent is selected from the group consisting of azide, dibenzocyclooctyne (DBCO), transcyclooctene, tetrazine, norbornene, and variants thereof, optionally, wherein the first click reagent is azide and the second click reagent is dibenzocyclooctyne (DBCO).

11. The method of claim 1, wherein the agent is selected from the group consisting of a small molecule, a nucleic acid, a protein or a peptide, and any combination thereof.

12. The method of claim 1, wherein the agent comprises a protein or a peptide, optionally, wherein the protein or peptide is selected from the group consisting of a transcriptional factor, a growth factor, a cytokine, an antibody, and a gene editing protein or peptide.

13. The method of claim 12, wherein the protein or peptide is:
(i) a transcriptional factor that modulates the expression of one or more genes; or
(ii) a gene editing protein or peptide that edits or modifies a gene or the genome of the cell, optionally, wherein the gene editing protein or peptide is selected from the group consisting of meganuclease, zinc finger nuclease (ZFN), transcription activator like effector-based nuclease (TALEN), and CRISPR associate protein (Cas), or wherein the protein or peptide is a CRISPR associate protein 9 (Cas9).

14. The method of claim 1, wherein the agent comprises a nucleic acid, optionally, wherein the nucleic acid encodes a protein or a peptide, or wherein the nucleic acid modulates the expression of a gene.

15. The method of claim 14, wherein the nucleic acid is selected from the group consisting of siRNA, shRNA, ribozyme RNA, iRNA, sgRNA, and miRNA.

16. The method of claim 1, wherein the cell is selected from the group consisting of a stem cell, a primary cell, a neural cell, and a fibroblast cell.

17. The method of claim 1, comprising contacting a cell coupled to an azide with the agent coupled to dibenzocyclooctyne (DBCO), thereby delivering the agent to the cell intracellularly, wherein the agent retain its structural integrity, function and/or activity while residing within the cell.

18. The method of claim 1, comprising delivering a CRISPR associated protein 9 (Cas9) to a cell intracellularly, comprising contacting a cell coupled to an azide with the Cas9 coupled to dibenzocyclooctyne (DBCO), thereby delivering the Cas9 to the cell intracellularly, wherein the Cas9 retains its structural integrity, function and/or activity while residing within the cell.

19. The method of claim 1, wherein the agent is delivered to the cell in vitro.

20. The method of claim 1, wherein the agent is delivered to the cell in vivo in a subject, comprising administering to the subject the cell coupled or to be coupled to the first click agent and the agent coupled to the second click agent.

21. The method of claim 20, wherein:
(i) the agent is administered to the subject prior to, concurrently with, or after the administration of the cell coupled to the click agent;
(ii) the cell is coupled in vitro and administered to the subject in the absence of a scaffold; or
(iii) the cell is coupled in vivo within a scaffold.

22. (canceled)

23. (canceled)

24. A method of editing or modifying a gene or the genome of a cell, comprising:
contacting the cell which is coupled to a first click reagent with a gene editing molecule coupled to a second click reagent;
wherein the second click reagent selectively reacts with the first click reagent coupled to the cell, thereby editing or modifying the gene or the genome of the cell.

25. The method of claim 14, wherein:
(i) the first click reagent is selected from the group consisting of azide, dibenzocyclooctyne (DBCO), transcyclooctene, tetrazine, norbornene, and variants thereof,
(ii) the first click reagent is azide and the second click reagent is dibenzocyclooctyne (DBCO);
(iii) the gene editing molecule is selected from the group consisting of meganuclease, zinc finger nuclease (ZFN), transcription activator-like effector-based nuclease (TALEN), and CRISPR associate protein (Cas);
(iv) the gene editing molecule is CRISPR associate protein 9 (Cas9);
(v) the gene editing molecule is a nucleic acid that encoding a protein or peptide;
(vi) the protein or peptide is selected from the group consisting of meganuclease, zinc finger nuclease (ZFN), transcription activator-like effector-based nuclease (TALEN), and CRISPR associate protein (Cas);
(vii) the protein or peptide is CRISPR associate protein 9 (Cas9);
(viii) the method further comprises contacting the cell with a single guide RNA (sgRNA);
(ix) the gene editing molecule contacts the cell in vitro;
(x) the gene editing molecule is targeted to the cell in vivo in a subject, comprising administering the subject the cell coupled or to be coupled to the first click agent and the gene editing molecule coupled to the second click agent;
(xi) the gene editing molecule is administered to the subject prior to, concurrently with, or after the administration of the cell coupled to the first click agent;
(xii) the cell is coupled in vitro and administered to the subject in the absence of a scaffold; and/or
(xiii) the cell is coupled in vivo within a scaffold.

26.-37. (canceled)

38. A method of inducing differentiation of a cell, comprising:
contacting the cell which is coupled with a first click reagent with a cell differentiation inducing agent coupled to a second click reagent;
wherein the second click reagent selectively reacts with the first click reagent coupled to the cell, thereby inducing the differentiation of the cell.

39. The method of claim 38, wherein:
(i) the cell differentiation inducing agent is present within the cell, and wherein the cell differentiation inducing agent retains its structural integrity, function and/or activity while residing within the cell;
(ii) the first click reagent is selected from the group consisting of azide, dibenzocyclooctyne (DBCO), transcyclooctene, tetrazine, norbornene, and variants thereof;
(iii) the first click reagent is azide and the second click reagent is dibenzocyclooctyne (DBCO);
(iv) the cell differentiation inducing agent is a transcriptional regulator or a gene editing molecule;
(v) the cell differentiation inducing agent is a gene editing molecule selected from the group consisting of meganuclease, zinc finger nuclease (ZFN), transcription activator-like effector-based nuclease (TALEN), and CRISPR associate protein (Cas);
(vi) the method further comprises contacting the cell with a single guide RNA (sgRNA);
(vii) the cell is selected from the group consisting of a stem cell, a primary cell, a neural cell, and a fibroblast cell;
(viii) the differentiation inducing agent contacts the cell in vitro;
(ix) the differentiation inducing agent is targeted to the cell in vivo in a subject, comprising administering the subject cell coupled or to be to the first click agent and the differentiation inducing agent coupled to the second click agent;
(x) the differentiation inducing agent is administered to the subject prior to, concurrently with, or after the administration of the cell coupled or to be coupled to the first click agent;
(xi) the cell is coupled in vitro and administered to the subject in the absence of a scaffold; and/or
(xii) the cell is coupled in vivo within a scaffold.

40.-50. (canceled)

51. A cell comprising
a glycoprotein coupled to a first click agent; and
an agent coupled to a second click agent,
wherein the glycoprotein is covalently linked to the agent through a selective reaction between the first click agent and the second click agent, wherein the glycoprotein-agent complex is located within the cell, and wherein the agent retains its structural integrity, function or activity.

52.-59. (canceled)
